SEQUENCE LISTING (26 sequences)

SEQ ID NO: 1 - DNA, 9653 bp, Homo sapiens
ctattggcagagctctagggtccctggcagtgtcctgccacacaccaccaatcatccgagctgttggcatgtggtgaggaccttttcccccttcctttttcctccccacagctgcaggagggggattctgcggggcggatgtcactttgctaggggcgggttcttcccacccccacactacatcttgttctagctccttcctatcccggatgtaggccattttggggttcctagtaggggtcacatcccggtaaaagaacattagggagcacctttgggcacccctgctcagcagccccctttcccccaactggttccctctctcctccatcagccaggcccccaccacctatgagggcctcgcggtccctgggtcctccgccacaaatgccccctttttccttggggctgggtgggaagggacagctttgggactttttaaagaaagaactttgctaggaaagtaagagacgaaccaaaaccaaaccaagaaaatgtcaagacctccgctggtccggaatctgcgctcggcctctggctctgttctgctcctctgccttgacttcacctaggaagctgcagccttcttaagccctctgcaactcccttgctcacctgctccctggctccccatgagggtgcatggggggcagtgctgccacttgcctttccagaggtgctgcagccatggctcttccttaccttcacctctggcccaggcctgttcctgaacccccagttactggaacccccaggatgatgagaagcgagggatgcaaaccaggaacccccagtctcatggggagaacggagcctcaccctggagctggggagatcgtctgggtcagtgcagctccagctacacatcataagccccagtggacaaggcagccaggaggagggtggggcagatcagggcagataaccagaaggaccgagtttggggcccagcatggcccatatgggtttaagatgcatggagaggaagcacagggaggacatggggacaggcccatcccctacccctgtgttctggacctcagcgtcccctccgtggcccctctggccctggcccagggatgccttctgcttcattgagcccatctgtttgagacttgctaacaccttcctcttttgtgactggaagtcacagggccaattgctaagccgtgcagtcacagagggaacacagagcctagttgtaaacggacagagacgagaggggcaagggaggacagtggatgacagggaagacgagtgggggcagagctgctcaggaccatggctgaggccatcacctatgcagatctgaggtttgtgaaggctcccctgaagaagagcatctccagccggttaggacagggtaagggggatgaggcctccccttgatcctgcatccccacttcccgccccacagatcccattcccaaatcccaccttggtcccttttctcttctctctagacccaggggctgatgatgatggggaaatcacctacgagaatgttcaagtgcccgcagtcctaggggtgccctcaagcttggcttcttctgtactaggggacaaagcaggtctggagagcctgggggatgtgtgtttgttggggggctgtgtcttgagctggggagtccacaatgtttttgttgttgttcttgagacagggtcttgttctgtcactcaggctggagtgcagtggtgtgatcatggctcactgcagcctcgacctcctgggctcaagtgatcctcctgccctggcctcccaaagtgttgggattgcaggcgtgcgcccccatgccatggccctcagtattcttgagagcacaataccctggggaaaggaggagggcggttcttaggaaacagaagaataggaacaggataaatgggagccaggggaggaagaaagattcctcccgcaaggtggagacccctttcggggagataagggctgcggagggatagggcagccctgtgggggatgagttctaatgggaaacggggagccatgggagggaaaagaggtgatgggagatggatgagagcaaagggggagagggtttggagccgacagataaggggcctctgaggaaacgataaagggagaactaggagagatgagggggtcccgggaaaagatacagtgagacccaggaggaagccgagagagtcccgggcagaggaggaactcaggagagtgacactgtgtcccagagcagctctagcaggcaacgtagggcagactggagtttcccagtggcgagggcgaagcgggtgcgcaggaggagaaggggcaacactcggggcttccggtctcacactggatgctcccctcgacagcggtcaagtcggagcagccaactgcgtcctggagagccgtgacgtcaccagctgtcgggcggattctcccctgtgagtgtgcctgtgcggggcagccgcggggccaagtgctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggtaacgcccccgagtccccaccagcactgattctggaacaccctctcccctacctgcgaaacaccccgctacccgacagaaaccaccatgcttgccccctcagctctgcaccgccctcctcttagggatgccttccccaggggtctcccccacggacctgtcccttcctctctacattctttacgggggctgactgccctcatccatcccactgtccccctaaggccgcacaacctgcctgcgatacctcctgctcggcctgctcctcacctgcctgctgttaggagtgaccgccatctgcctgggagtgcgctgtgagtaaggctgtccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcaggtatccgctctggggagaggagagaagggagggatggattggagcaaggagatagtagtggcaggagtctatcacccaggggcctgtgaggtgttcaggaaaggggcagtgaaccagagtggtggctgacatgagctgttcagccttggccaacagagagaaggaccaagctgcatactagatttagggggacccacccagctccaaggctcatcctgtaggggagaggaggatggcagccagcagtccaaatcacactgtccaaggtttcttccggttgatatcagtgacaaggatctgggaacgcccccaaatttggatgtgaacttggcggtttgcacatctttctaaagacagggacctcatttttaattaattttcgttatagctgagatctccccaaagggtaagacttactggctgacatggagtgcaacctggtggtggggcaaggcagtgaggcgctcagcactcacagtgaggtggagctgggagttgtggtgtagggaactaggatggggtgtccattcattttgaaagcaggaccccagccagtaagtggagatggggaaagggacacataaaagaggcaaggctgtatcaaaacaggctgagggtctaggcaaggtgccacgcctaaggggactggggtgtttttgggctatgatcaggtttaggctctgtctagcttgggggtcatctgggtgatctccccaggcataagacagcaagattatgtgcagacaccccttgccctcatttccttaaccttaccctctgtgaccatcagttccatgccagggctgctcttagcagcatgttagacctctacctcccaaattgctcttccggcgaaaggatggtttcagacttccactctgatcacagctgcccaccactcctgcaggttttccccacacttttttgatctcatcaaggcctccaatctgctgactcccctttttctcccttcccatcagtccaggcctccaatcaactgattcccctttttctctcttcccatcagcttctcctgtccacacttctctccctacctaacatgggtttggagcccatcacttcaactactcacttatttttttaaatagagacaaggtctcactatgtttccctggccggtatcgaactcctggactcaagcgatcctcctgcctcagcctcccaaagtgctgagattacagtcgtgagccaccgcacctggctcactcacttctttgttccattatccttctagtgtacctgatgggcaaaatttgaactctggatgaacccaactgcccatggtcagcatcaaggtgtgacaaattgtggaatcattctgctcagtgccactgagactctagtctctcagcactgcctggcaacctttgcaatggtcagcttgctctccatagtaactccttctccctcactttcagcagatgtcctcatcttccactctttcagagaggaattcccttcactttccatcaccaagtctatatatccatatggatttttgcccatcctcatcttttgcaaggatatctgcaacagctttctaaatgtttttcctactcactcctctccctgcagtccattcttcatattggagccagggtaatcttttacaaacaaaaatctgatcgtgtcacccaatctacagcattccagttaattgattacataaacagttatggaatacataacatataatatgttctagcactgtgcccaggcacttgggatatagctagtgaacaaaattcctctatgtaaaaccctacactgactttctcttcctcataatataaaaataaaaatccttaacatggcttccatcactcctgcctacctttcagtgtgccacttatctcctcaccctcttccatttggccatacagatttctttttttttttttttgaggcggagtctcgctctgtgagactgaagtgcactcccaggctggagtgcagtggtgtgatctgggctcactgcaacctctgcctcctggattcaagctatccttttgcctcagcctcccgagtaactgggattataggcatgcgccatggtgcctgggtattttttggtatttttagtagaggtggggtttcaccatgttgaccaggctggtcttaaactcctgacttcaggtgatccaccccgcctcggcctcccaaagtgctgggattacagaagtgagccattgtgcccagcagggatttctttttattcctttaagttgccataggacagtagctcaagggagtagcagggtggggacatgagcatatgtagagagaagagagggagctagtggagagggaatgatttttgagtgatgaaggtaggactgatggaacaaagcaatggagagaggtggcctgggatcagaagtgcacattgggatgtaggatgttgaagaaagagggatatttatttcttatggatagtcaggaaaagaagaacagagaagagaaattttgaggtggagaggagggaaaaggagagagctcagtttctgcaaaacagtacacaacattttctgcttgtaggaaattggagagggacggagggggccagtgctggaggagtggaggatgtgcagtgaggggtgctttagggagtggaggagatgggcccaagagatgttaggtaaagtgtcagggtggactgcaattgttcagaaattggggtaataaaatcagccctattaagacagacctcccagattgctaagaagtaatgttgttgaatgttaaagtctttttactaagtaaggacaggtatggtgtttccaggggatgcaaaagaaagattgcaaagatcatagaagtccatgaaggtgctgagattagctctgggggaatattgttagcaaaagattgctgagtagtcactttcatcccaactgttgctgctatcacactctttctgctttttagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcagtaagcatatccttactcacagactgcagtgggtactattaggtaaagggacatatgtcatcaagtcaggaatggcagtgcacacatggctggggtggccattccttttccatggaagaaactgagaatcataatactcttgacctctgagcttggaaacaacctctgcatcttctcagcacagtggtccaggcagcccctagtaactgattagagttggcatgagaatggaaacttaaccatatctacatttttttttttttgagacagagtctcactctgtcacccaggctggagtgcacttgcataatctcagctcactgcaacctccgcctcctgggttcatgcaattctcccgtctcagcctcctgagtagctgggactacagtcacatgccaccacgcccagctaatttttgtatttttaatagagtcggggtttcaccatattggtcaggctggtctctaactcctggcctcaggtgatccacctgcttcagtctcccaaagtgctgggattataggtgtgaaccactgcgcccggcctctactttaatgtgtatctcatctcagcttgctggtcagctccctgggctgcttgacaggttcctggactcttgtggcctctcaggcagagaggtcccacacctggtgggctggattgtagggattagttccttggggccagaacagcacacagaatgactcccctccaaaggctggcagagattcttactcagctggaaggcaaatttagcagtgagcctgcatctgcagggtggcacttgatggattcagggacaagttaccaaagagctgtcatatatacagcccccttgccaaagctgaggcctccctagggtgtgcattttcttccatatttctcatcactctccatgtatctctggtctctctgaatctcccctcagcactcttactacttcttaaattcactgttgccaaatggtggttcagggaattcatattggactggcctcagctctaacaaggattggaagttgactgatgatacacaacgcactaggtaagtttgttaggacttctgggtattttcaactccataatcccctccataaagacatggtaatcagatttccttcccacctggtcccctcaggactctcccataaatcaacactgtaattgcctatttgtctgtttcctctatttgattaataacttcttagggcaatcttattctccattctatttccacctagaagtccctggcacatacttttttgtttcagtaactactaggttgggacctacgctatcttttagggggagacattaaggatttagttaaggctccacagctgatgacagcagcgtttctccatccttcgagaaggccacccaaactgaggttgtactttctcagtagcggccacttggggcactagagctgcctcagtgaagtacgcaagggagcagcgggggtaccatcgaagctccacttcactgtgatcaagacagcagtggggtaccattgaggatcaaccctagggaccctgaaacagggtggcaacctccttatgttcctcaattggccttcccatcttttttttttttttgagatggagtctcgctctgtcgcccagactggagtgcagtggtgcaatctcagctcactgcaagctccgcctcaggggttcacaccattctcctgcctcagcctccccagcagctgggactacaggcgcacaccgccatgcccggctaattttttgtatttttagtagagacggggtttcaccgtgttagtcaggatggtctcgatgtcctgaccttgtgatccacccgccttggcctcccaaagtgctgggattgcaggcatgagccaccgtgcccagcctggccttcccatctttaagatctcgagttatttttccctctgctggttctaaagtctgttttagttgaactccagacatctgtcctgaaccatgcccagatgtggccttaggcaggcagggtgagttaggatttaggttttatacaggggaaggggagagaagggagggtgggatatgtccagagcatatcccagctctaccttactactttttaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttggtaagagtgagggatggggctgaggcatggggagtccaggggcagagggactcagagcagggactcagagcaggggcccaggggcccgagagaaaacttgtacctgggatgaaagttcctccacagcctccagttgcttggtctgcttgttagggtggggtgggcagggagggacaggtaaggcctacaacttatgggaggctggtggtgtggctgtcgagagctgaggaggagatggggtgagtgggatgagggttcagttaagtcaaccatggagctcactgatactttacttttccagacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattgcacgtgttagtctcatgtgtcagcaatgctgtgtctggttcagtgat

SEQ ID NO: 2 - DNA, 1532 bp, Homo sapiens
agtcacagagggaacacagagcctagttgtaaacggacagagacgagaggggcaagggaggacagtggatgacagggaagacgagtgggggcagagctgctcaggaccatggctgaggccatcacctatgcagatctgaggtttgtgaaggctcccctgaagaagagcatctccagccggttaggacaggacccaggggctgatgatgatggggaaatcacctacgagaatgttcaagtgcccgcagtcctaggggtgccctcaagcttggcttcttctgtactaggggacaaagcagcggtcaagtcggagcagccaactgcgtcctggagagccgtgacgtcaccagctgtcgggcggattctcccctgccgcacaacctgcctgcgatacctcctgctcggcctgctcctcacctgcctgctgttaggagtgaccgccatctgcctgggagtgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattg

SEQ ID NO: 3 - protein, 359 aa, Homo sapiens
MAEAITYADLRFVKAPLKKSISSRLGQDPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 4 - DNA, 9373 bp, Homo sapiens
ggccaccccagggtctggtccctgacgacgcgcagtgagggccccgccgctaccccagcagtcgcctcccaagttcgcggaacgcagctgaccggctccctctggactgggtgacatgactgctcccaagcagtcgtttgtaaactgagtttctgtaaaacaattttatttttcatatgtgactgtagcggggtatgatttgaactttgttttccgtcccccagcccggattctctgtcttctcctgtacagccgttccgttttcttacctcgtctccgtcaccgaggccctcagccctgaacacaaggactgggcagtttccctattgattcctgaacctggaacttaagacatcttccgaggggcccccccttgccacaccctctagctgatcgactcacaaatacctgtgatttctctccccgtctccacctccaggcctgttcctgaacccccagttactggaacccccaggatgatgagaagcgagggatgcaaaccaggaacccccagtctcatggggagaacggagcctcaccctggagctggggagatcgtctgggtcagtgcagctccagctacacatcataagccccagtggacaaggcagccaggaggagggtggggcagatcagggcagataaccagaaggaccgagtttggggcccagcatggcccatatgggtttaagatgcatggagaggaagcacagggaggacatggggacaggcccatcccctacccctgtgttctggacctcagcgtcccctccgtggcccctctggccctggcccagggatgccttctgcttcattgagcccatctgtttgagacttgctaacaccttcctcttttgtgactggaagtcacagggccaattgctaagccgtgcagtcacagagggaacacagagcctagttgtaaacggacagagacgagaggggcaagggaggacagtggatgacagggaagacgagtgggggcagagctgctcaggaccatggctgaggccatcacctatgcagatctgaggtttgtgaaggctcccctgaagaagagcatctccagccggttaggacagggtaagggggatgaggcctccccttgatcctgcatccccacttcccgccccacagatcccattcccaaatcccaccttggtcccttttctcttctctctagacccaggggctgatgatgatggggaaatcacctacgagaatgttcaagtgcccgcagtcctaggggtgccctcaagcttggcttcttctgtactaggggacaaagcaggtctggagagcctgggggatgtgtgtttgttggggggctgtgtcttgagctggggagtccacaatgtttttgttgttgttcttgagacagggtcttgttctgtcactcaggctggagtgcagtggtgtgatcatggctcactgcagcctcgacctcctgggctcaagtgatcctcctgccctggcctcccaaagtgttgggattgcaggcgtgcgcccccatgccatggccctcagtattcttgagagcacaataccctggggaaaggaggagggcggttcttaggaaacagaagaataggaacaggataaatgggagccaggggaggaagaaagattcctcccgcaaggtggagacccctttcggggagataagggctgcggagggatagggcagccctgtgggggatgagttctaatgggaaacggggagccatgggagggaaaagaggtgatgggagatggatgagagcaaagggggagagggtttggagccgacagataaggggcctctgaggaaacgataaagggagaactaggagagatgagggggtcccgggaaaagatacagtgagacccaggaggaagccgagagagtcccgggcagaggaggaactcaggagagtgacactgtgtcccagagcagctctagcaggcaacgtagggcagactggagtttcccagtggcgagggcgaagcgggtgcgcaggaggagaaggggcaacactcggggcttccggtctcacactggatgctcccctcgacagcggtcaagtcggagcagccaactgcgtcctggagagccgtgacgtcaccagctgtcgggcggattctcccctgtgagtgtgcctgtgcggggcagccgcggggccaagtgctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggtaacgcccccgagtccccaccagcactgattctggaacaccctctcccctacctgcgaaacaccccgctacccgacagaaaccaccatgcttgccccctcagctctgcaccgccctcctcttagggatgccttccccaggggtctcccccacggacctgtcccttcctctctacattctttacgggggctgactgccctcatccatcccactgtccccctaaggccgcacaacctgcctgcgatacctcctgctcggcctgctcctcacctgcctgctgttaggagtgaccgccatctgcctgggagtgcgctgtgagtaaggctgtccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctcagcaacatggagaacagactgaagcccttcttcacatgcggctcagcaggtatccgctctggggagaggagagaagggagggatggattggagcaaggagatagtagtggcaggagtctatcacccaggggcctgtgaggtgttcaggaaaggggcagtgaaccagagtggtggctgacatgagctgttcagccttggccaacagagagaaggaccaagctgcatactagatttagggggacccacccagctccaaggctcatcctgtaggggagaggaggatggcagccagcagtccaaatcacactgtccaaggtttcttccggttgatatcagtgacaaggatctgggaacgcccccaaatttggatgtgaacttggcggtttgcacatctttctaaagacagggacctcatttttaattaattttcgttatagctgagatctccccaaagggtaagacttactggctgacatggagtgcaacctggtggtggggcaaggcagtgaggcgctcagcactcacagtgaggtggagctgggagttgtggtgtagggaactaggatggggtgtccattcattttgaaagcaggaccccagccagtaagtggagatggggaaagggacacataaaagaggcaaggctgtatcaaaacaggctgagggtctaggcaaggtgccacgcctaaggggactggggtgtttttgggctatgatcaggtttaggctctgtctagcttgggggtcatctgggtgatctccccaggcataagacagcaagattatgtgcagacaccccttgccctcatttccttaaccttaccctctgtgaccatcagttccatgccagggctgctcttagcagcatgttagacctctacctcccaaattgctcttccggcgaaaggatggtttcagacttccactctgatcacagctgcccaccactcctgcaggttttccccacacttttttgatctcatcaaggcctccaatctgctgactcccctttttctcccttcccatcagtccaggcctccaatcaactgattcccctttttctctcttcccatcagcttctcctgtccacacttctctccctacctaacatgggtttggagcccatcacttcaactactcacttatttttttaaatagagacaaggtctcactatgtttccctggccggtatcgaactcctggactcaagcgatcctcctgcctcagcctcccaaagtgctgagattacagtcgtgagccaccgcacctggctcactcacttctttgttccattatccttctagtgtacctgatgggcaaaatttgaactctggatgaacccaactgcccatggtcagcatcaaggtgtgacaaattgtggaatcattctgctcagtgccactgagactctagtctctcagcactgcctggcaacctttgcaatggtcagcttgctctccatagtaactccttctccctcactttcagcagatgtcctcatcttccactctttcagagaggaattcccttcactttccatcaccaagtctatatatccatatggatttttgcccatcctcatcttttgcaaggatatctgcaacagctttctaaatgtttttcctactcactcctctccctgcagtccattcttcatattggagccagggtaatcttttacaaacaaaaatctgatcgtgtcacccaatctacagcattccagttaattgattacataaacagttatggaatacataacatataatatgttctagcactgtgcccaggcacttgggatatagctagtgaacaaaattcctctatgtaaaaccctacactgactttctcttcctcataatataaaaataaaaatccttaacatggcttccatcactcctgcctacctttcagtgtgccacttatctcctcaccctcttccatttggccatacagatttctttttttttttttttgaggcggagtctcgctctgtgagactgaagtgcactcccaggctggagtgcagtggtgtgatctgggctcactgcaacctctgcctcctggattcaagctatccttttgcctcagcctcccgagtaactgggattataggcatgcgccatggtgcctgggtattttttggtatttttagtagaggtggggtttcaccatgttgaccaggctggtcttaaactcctgacttcaggtgatccaccccgcctcggcctcccaaagtgctgggattacagaagtgagccattgtgcccagcagggatttctttttattcctttaagttgccataggacagtagctcaagggagtagcagggtggggacatgagcatatgtagagagaagagagggagctagtggagagggaatgatttttgagtgatgaaggtaggactgatggaacaaagcaatggagagaggtggcctgggatcagaagtgcacattgggatgtaggatgttgaagaaagagggatatttatttcttatggatagtcaggaaaagaagaacagagaagagaaattttgaggtggagaggagggaaaaggagagagctcagtttctgcaaaacagtacacaacattttctgcttgtaggaaattggagagggacggagggggccagtgctggaggagtggaggatgtgcagtgaggggtgctttagggagtggaggagatgggcccaagagatgttaggtaaagtgtcagggtggactgcaattgttcagaaattggggtaataaaatcagccctattaagacagacctcccagattgctaagaagtaatgttgttgaatgttaaagtctttttactaagtaaggacaggtatggtgtttccaggggatgcaaaagaaagattgcaaagatcatagaagtccatgaaggtgctgagattagctctgggggaatattgttagcaaaagattgctgagtagtcactttcatcccaactgttgctgctatcacactctttctgctttttagacacctgctgtctgtcgggatggataatgcatcagaaaagctgcttttacatctcacttacttcaaaaaattggcaggagagccaaaaacaatgtgaaactctgtcttccaagctggccacattcagtgaaatttatccacaatcagtaagcatatccttactcacagactgcagtgggtactattaggtaaagggacatatgtcatcaagtcaggaatggcagtgcacacatggctggggtggccattccttttccatggaagaaactgagaatcataatactcttgacctctgagcttggaaacaacctctgcatcttctcagcacagtggtccaggcagcccctagtaactgattagagttggcatgagaatggaaacttaaccatatctacatttttttttttttgagacagagtctcactctgtcacccaggctggagtgcacttgcataatctcagctcactgcaacctccgcctcctgggttcatgcaattctcccgtctcagcctcctgagtagctgggactacagtcacatgccaccacgcccagctaatttttgtatttttaatagagtcggggtttcaccatattggtcaggctggtctctaactcctggcctcaggtgatccacctgcttcagtctcccaaagtgctgggattataggtgtgaaccactgcgcccggcctctactttaatgtgtatctcatctcagcttgctggtcagctccctgggctgcttgacaggttcctggactcttgtggcctctcaggcagagaggtcccacacctggtgggctggattgtagggattagttccttggggccagaacagcacacagaatgactcccctccaaaggctggcagagattcttactcagctggaaggcaaatttagcagtgagcctgcatctgcagggtggcacttgatggattcagggacaagttaccaaagagctgtcatatatacagcccccttgccaaagctgaggcctccctagggtgtgcattttcttccatatttctcatcactctccatgtatctctggtctctctgaatctcccctcagcactcttactacttcttaaattcactgttgccaaatggtggttcagggaattcatattggactggcctcagctctaacaaggattggaagttgactgatgatacacaacgcactaggtaagtttgttaggacttctgggtattttcaactccataatcccctccataaagacatggtaatcagatttccttcccacctggtcccctcaggactctcccataaatcaacactgtaattgcctatttgtctgtttcctctatttgattaataacttcttagggcaatcttattctccattctatttccacctagaagtccctggcacatacttttttgtttcagtaactactaggttgggacctacgctatcttttagggggagacattaaggatttagttaaggctccacagctgatgacagcagcgtttctccatccttcgagaaggccacccaaactgaggttgtactttctcagtagcggccacttggggcactagagctgcctcagtgaagtacgcaagggagcagcgggggtaccatcgaagctccacttcactgtgatcaagacagcagtggggtaccattgaggatcaaccctagggaccctgaaacagggtggcaacctccttatgttcctcaattggccttcccatcttttttttttttttgagatggagtctcgctctgtcgcccagactggagtgcagtggtgcaatctcagctcactgcaagctccgcctcaggggttcacaccattctcctgcctcagcctccccagcagctgggactacaggcgcacaccgccatgcccggctaattttttgtatttttagtagagacggggtttcaccgtgttagtcaggatggtctcgatgtcctgaccttgtgatccacccgccttggcctcccaaagtgctgggattgcaggcatgagccaccgtgcccagcctggccttcccatctttaagatctcgagttatttttccctctgctggttctaaagtctgttttagttgaactccagacatctgtcctgaaccatgcccagatgtggccttaggcaggcagggtgagttaggatttaggttttatacaggggaaggggagagaagggagggtgggatatgtccagagcatatcccagctctaccttactactttttaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttggtaagagtgagggatggggctgaggcatggggagtccaggggcagagggactcagagcagggactcagagcaggggcccaggggcccgagagaaaacttgtacctgggatgaaagttcctccacagcctccagttgcttggtctgcttgttagggtggggtgggcagggagggacaggtaaggcctacaacttatgggaggctggtggtgtggctgtcgagagctgaggaggagatggggtgagtgggatgagggttcagttaagtcaaccatggagctcactgatactttacttttccagacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattgcacgtgttagtctcatgtgtcagcaatgctgtgtctggttcagtgat

SEQ ID NO: 5 - DNA, 1917 bp, Homo sapiens
ggccaccccagggtctggtccctgacgacgcgcagtgagggccccgccgctaccccagcagtcgcctcccaagttcgcggaacgcagctgaccggctccctctggactgggtgacatgactgctcccaagcagtcgtttgtaaactgagtttctgtaaaacaattttatttttcatatgtgactgtagcggggtatgatttgaactttgttttccgtcccccagcccggattctctgtcttctcctgtacagccgttccgttttcttacctcgtctccgtcaccgaggccctcagccctgaacacaaggactgggcagtttccctattgattcctgaacctggaacttaagacatcttccgaggggcccccccttgccacaccctctagctgatcgactcacaaatacctgtgatttctctccccgtctccacctccaggcctgttcctgaacccccagttactggaacccccaggatgatgagaagcgagggatgcaaaccaggaacccccagtctcatggggagaacggagcctcaccctggagctggggagatcgtctggacccaggggcggacccaggggctgatgatgatggggaaatcacctacgagaatgttcaagtgcccgcagtcctaggggtgccctcaagcttggcttcttctgtactaggggacaaagcagcggtcaagtcggagcagccaactgcgtcctggagagccgtgacgtcaccagctgtcgggcggattctcccctgccgcacaacctgcctgcgatacctcctgctcggcctgctcctcacctgcctgctgttaggagtgaccgccatctgcctgggagtgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctcagcaacatggagaacagactgaagcccttcttcacatgcggctcagcagacacctgctgtctgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattg

SEQ ID NO: 6 - protein, 359 aa, Homo sapiens
MQTRNPQSHGENGASPWSWGDRLDPGADPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 7 - DNA, 1387 bp, Homo sapiens
gctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggccgcacaacctgcctgcgatacctcctgctcggcctgctcctcacctgcctgctgttaggagtgaccgccatctgcctgggagtgcgctatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattg

SEQ ID NO: 8 - protein, 42 aa, Homo sapiens
GSRLSRLRMALGPAGSAPSGINPGAVLPTPGLPCHIHLHPVR

SEQ ID NO: 9 - DNA, 9592 bp, Homo sapiens
ctattggcagagctctagggtccctggcagtgtcctgccacacaccaccaatcatccgagctgttggcatgtggtgaggaccttttcccccttcctttttcctccccacagctgcaggagggggattctgcggggcggatgtcactttgctaggggcgggttcttcccacccccacactacatcttgttctagctccttcctatcccggatgtaggccattttggggttcctagtaggggtcacatcccggtaaaagaacattagggagcacctttgggcacccctgctcagcagccccctttcccccaactggttccctctctcctccatcagccaggcccccaccacctatgagggcctcgcggtccctgggtcctccgccacaaatgccccctttttccttggggctgggtgggaagggacagctttgggactttttaaagaaagaactttgctaggaaagtaagagacgaaccaaaaccaaaccaagaaaatgtcaagacctccgctggtccggaatctgcgctcggcctctggctctgttctgctcctctgccttgacttcacctaggaagctgcagccttcttaagccctctgcaactcccttgctcacctgctccctggctccccatgagggtgcatggggggcagtgctgccacttgcctttccagaggtgctgcagccatggctcttccttaccttcacctctggcccaggcctgttcctgaacccccagttactggaacccccaggatgatgagaagcgagggatgcaaaccaggaacccccagtctcatggggagaacggagcctcaccctggagctggggagatcgtctgggtcagtgcagctccagctacacatcataagccccagtggacaaggcagccaggaggagggtggggcagatcagggcagataaccagaaggaccgagtttggggcccagcatggcccatatgggtttaagatgcatggagaggaagcacagggaggacatggggacaggcccatcccctacccctgtgttctggacctcagcgtcccctccgtggcccctctggccctggcccagggatgccttctgcttcattgagcccatctgtttgagacttgctaacaccttcctcttttgtgactggaagtcacagggccaattgctaagccgtgcagtcacagagggaacacagagcctagttgtaaacggacagagacgagaggggcaagggaggacagtggatgacagggaagacgagtgggggcagagctgctcaggaccatggctgaggccatcacctatgcagatctgaggtttgtgaaggctcccctgaagaagagcatctccagccggttaggacagggtaagggggatgaggcctccccttgatcctgcatccccacttcccgccccacagatcccattcccaaatcccaccttggtcccttttctcttctctctagacccaggggctgatgatgatggggaaatcacctacgagaatgttcaagtgcccgcagtcctaggggtgccctcaagcttggcttcttctgtactaggggacaaagcaggtctggagagcctgggggatgtgtgtttgttggggggctgtgtcttgagctggggagtccacaatgtttttgttgttgttcttgagacagggtcttgttctgtcactcaggctggagtgcagtggtgtgatcatggctcactgcagcctcgacctcctgggctcaagtgatcctcctgccctggcctcccaaagtgttgggattgcaggcgtgcgcccccatgccatggccctcagtattcttgagagcacaataccctggggaaaggaggagggcggttcttaggaaacagaagaataggaacaggataaatgggagccaggggaggaagaaagattcctcccgcaaggtggagacccctttcggggagataagggctgcggagggatagggcagccctgtgggggatgagttctaatgggaaacggggagccatgggagggaaaagaggtgatgggagatggatgagagcaaagggggagagggtttggagccgacagataaggggcctctgaggaaacgataaagggagaactaggagagatgagggggtcccgggaaaagatacagtgagacccaggaggaagccgagagagtcccgggcagaggaggaactcaggagagtgacactgtgtcccagagcagctctagcaggcaacgtagggcagactggagtttcccagtggcgagggcgaagcgggtgcgcaggaggagaaggggcaacactcggggcttccggtctcacactggatgctcccctcgacagcggtcaagtcggagcagccaactgcgtcctggagagccgtgacgtcaccagctgtcgggcggattctcccctgtgagtgtgcctgtgcggggcagccgcggggccaagtgctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggtaacgcccccgagtccccaccagcactgattctggaacaccctctcccctacctgcgaaacaccccgctacccgacagaaaccaccatgcttgccccctcagctctgcaccgccctcctcttagggatgccttccccaggggtctcccccacggacctgtcccttcctctctacattctttacgggggctgactgccctcatccatcccactgtccccctaaggccgcacaacctgcctgcgatacctcctgctcggcctgctcctcacctgcctgctgttaggagtgaccgccatctgcctgggagtgcgctgtgagtaaggctgaccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcaggtatccgctctggggagaggagagaagggagggatggattggagcaaggagatagtagtggcaggagtctatcacccaggggcctgtgaggtgttcaggaaaggggcagtgaaccagagtggtggctgacatgagctgttcagccttggccaacagagagaaggaccaagctgcatactagatttagggggacccacccagctccaaggctcatcctgtaggggagaggaggatggcagccagcagtccaaatcacactgtccaaggtttcttccggttgatatcagtgacaaggatctgggaacgcccccaaatttggatgtgaacttggcggtttgcacatctttctaaagacagggacctcatttttaattaattttcgttatagctgagatctccccaaagggtaagacttactggctgacatggagtgcaacctggtggtggggcaaggcagtgaggcgctcagcactcacagtgaggtggagctgggagttgtggtgtagggaactaggatggggtgtccattcattttgaaagcaggaccccagccagtaagtggagatggggaaagggacacataaaagaggcaaggctgtatcaaaacaggctgagggtctaggcaaggtgccacgcctaaggggactggggtgtttttgggctatgatcaggtttaggctctgtctagcttgggggtcatctgggtgatctccccaggcataagacagcaagattatgtgcagacaccccttgccctcatttccttaaccttaccctctgtgaccatcagttccatgccagggctgctcttagcagcatgttagacctctacctcccaaattgctcttccggcgaaaggatggtttcagacttccactctgatcacagctgcccaccactcctgcaggttttccccacacttttttgatctcatcaaggcctccaatctgctgactcccctttttctcccttcccatcagtccaggcctccaatcaactgattcccctttttctctcttcccatcagcttctcctgtccacacttctctccctacctaacatgggtttggagcccatcacttcaactactcacttatttttttaaatagagacaaggtctcactatgtttccctggccggtatcgaactcctggactcaagcgatcctcctgcctcagcctcccaaagtgctgagattacagtcgtgagccaccgcacctggctcactcacttctttgttccattatccttctagtgtacctgatgggcaaaatttgaactctggatgaacccaactgcccatggtcagcatcaaggtgtgacaaattgtggaatcattctgctcagtgccactgagactctagtctctcagcactgcctggcaacctttgcaatggtcagcttgctctccatagtaactccttctccctcactttcagcagatgtcctcatcttccactctttcagagaggaattcccttcactttccatcaccaagtctatatatccatatggatttttgcccatcctcatcttttgcaaggatatctgcaacagctttctaaatgtttttcctactcactcctctccctgcagtccattcttcatattggagccagggtaatcttttacaaacaaaaatctgatcgtgtcacccaatctacagcattccagttaattgattacataaacagttatggaatacataacatataatatgttctagcactgtgcccaggcacttgggatatagctagtgaacaaaattcctctatgtaaaaccctacactgactttctcttcctcataatataaaaataaaaatccttaacatggcttccatcactcctgcctacctttcagtgtgccacttatctcctcaccctcttccatttggccatacagatttctttttttttttttttgaggcggagtctcgctctgtgagactgaagtgcactcccaggctggagtgcagtggtgtgatctgggctcactgcaacctctgcctcctggattcaagctatccttttgcctcagcctcccgagtaactgggattataggcatgcgccatggtgcctgggtattttttggtatttttagtagaggtggggtttcaccatgttgaccaggctggtcttaaactcctgacttcaggtgatccaccccgcctcggcctcccaaagtgctgggattacagaagtgagccattgtgcccagcagggatttctttttattcctttaagttgccataggacagtagctcaagggagtagcagggtggggacatgagcatatgtagagagaagagagggagctagtggagagggaatgatttttgagtgatgaaggtaggactgatggaacaaagcaatggagagaggtggcctgggatcagaagtgcacattgggatgtaggatgttgaagaaagagggatatttatttcttatggatagtcaggaaaagaagaacagagaagagaaattttgaggtggagaggagggaaaaggagagagctcagtttctgcaaaacagtacacaacattttctgcttgtaggaaattggagagggacggagggggccagtgctggaggagtggaggatgtgcagtgaggggtgctttagggagtggaggagatgggcccaagagatgttaggtaaagtgtcagggtggactgcaattgttcagaaattggggtaataaaatcagccctattaagacagacctcccagattgctaagaagtaatgttgttgaatgttaaagtctttttactaagtaaggacaggtatggtgtttccaggggatgcaaaagaaagattgcaaagatcatagaagtccatgaaggtgctgagattagctctgggggaatattgttagcaaaagattgctgagtagtcactttcatcccaactgttgctgctatcacactctttctgctttttagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcagtaagcatatccttactcacagactgcagtgggtactattaggtaaagggacatatgtcatcaagtcaggaatggcagtgcacacatggctggggtggccattccttttccatggaagaaactgagaatcataatactcttgacctctgagcttggaaacaacctctgcatcttctcagcacagtggtccaggcagcccctagtaactgattagagttggcatgagaatggaaacttaaccatatctacatttttttttttttgagacagagtctcactctgtcacccaggctggagtgcacttgcataatctcagctcactgcaacctccgcctcctgggttcatgcaattctcccgtctcagcctcctgagtagctgggactacagtcacatgccaccacgcccagctaatttttgtatttttaatagagtcggggtttcaccatattggtcaggctggtctctaactcctggcctcaggtgatccacctgcttcagtctcccaaagtgctgggattataggtgtgaaccactgcgcccggcctctactttaatgtgtatctcatctcagcttgctggtcagctccctgggctgcttgacaggttcctggactcttgtggcctctcaggcagagaggtcccacacctggtgggctggattgtagggattagttccttggggccagaacagcacacagaatgactcccctccaaaggctggcagagattcttactcagctggaaggcaaatttagcagtgagcctgcatctgcagggtggcacttgatggattcagggacaagttaccaaagagctgtcatatatacagcccccttgccaaagctgaggcctccctagggtgtgcattttcttccatatttctcatcactctccatgtatctctggtctctctgaatctcccctcagcactcttactacttcttaaattcactgttgccaaatggtggttcagggaattcatattggactggcctcagctctaacaaggattggaagttgactgatgatacacaacgcactaggtaagtttgttaggacttctgggtattttcaactccataatcccctccataaagacatggtaatcagatttccttcccacctggtcccctcaggactctcccataaatcaacactgtaattgcctatttgtctgtttcctctatttgattaataacttcttagggcaatcttattctccattctatttccacctagaagtccctggcacatacttttttgtttcagtaactactaggttgggacctacgctatcttttagggggagacattaaggatttagttaaggctccacagctgatgacagcagcgtttctccatccttcgagaaggccacccaaactgaggttgtactttctcagtagcggccacttggggcactagagctgcctcagtgaagtacgcaagggagcagcgggggtaccatcgaagctccacttcactgtgatcaagacagcagtggggtaccattgaggatcaaccctagggaccctgaaacagggtggcaacctccttatgttcctcaattggccttcccatcttttttttttttttgagatggagtctcgctctgtcgcccagactggagtgcagtggtgcaatctcagctcactgcaagctccgcctcaggggttcacaccattctcctgcctcagcctccccagcagctgggactacaggcgcacaccgccatgcccggctaattttttgtatttttagtagagacggggtttcaccgtgttagtcaggatggtctcgatgtcctgaccttgtgatccacccgccttggcctcccaaagtgctgggattgcaggcatgagccaccgtgcccagcctggccttcccatctttaagatctcgagttatttttccctctgctggttctaaagtctgttttagttgaactccagacatctgtcctgaaccatgcccagatgtggccttaggcaggcagggtgagttaggatttaggttttatacaggggaaggggagagaagggagggtgggatatgtccagagcatatcccagctctaccttactactttttaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttggtaagagtgagggatggggctgaggcatggggagtccaggggcagagggactcagagcagggactcagagcaggggcccaggggcccgagagaaaacttgtacctgggatgaaagttcctccacagcctccagttgcttggtctgcttgttagggtggggtgggcagggagggacaggtaaggcctacaacttatgggaggctggtggtgtggctgtcgagagctgaggaggagatggggtgagtgggatgagggttcagttaagtcaaccatggagctcactgatactttacttttccagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattgcacgtgttagtctcatgtgtcagcaatgctgtgtctggttcagtgat

SEQ ID NO: 10 - DNA, 1551 bp, Homo sapiens
  misc_feature: n is a or g or c or t/u, unknown, or other
ccgcagtcctaggggtgccctcaagcttggcttcttctgtactaggggacaaagcagcggtcaagtcggagcagccaactgcgtcctggagagccgtgacgtcaccagctgtcgggcggattctcccctgccgcacaacctgcctgcgatacctcctgctcggcctgctcctcacctgcctgctgttaggagtgaccgccatctgcctgggagtgcgctgtgagtaaggctgaccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattg

SEQ ID NO: 11 - DNA, 1802 bp, Homo sapiens
tggcagtcacagagggaacacagagcctagttgtaaacggacagagacgagaggggcaagggaggacagtggatgacagggaagacgagtgggggcagagctgctcaggaccatggctgaggccatcacctatgcagatctgagacccaggggctgatgatgatggggaaatcacctacgagaatgttcaagtgcccgcagtcctaggggtgccctcaagcttggcttcttctgtactaggggacaaagcagcggtcaagtcggagcagccaactgcgtcctggagagccgtgacgtcaccagctgtcgggcggattctcccctgccgcacaacctgcctgcgatacctcctgctcggcctgctcctcacctgcctgctgttaggagtgaccgccatctgcctgggagtgcgctgtgagtaaggctgtccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcagacacctgctgtctgtcgggatggataatgcatcagaaaagctgcttttacatctcacttacttcaaaaaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaac

SEQ ID NO: 12 - DNA, 423 bp, Homo sapiens
aataccctccgaccaccacaccgacagctctcgactcctcctctaccccactcaccctactcccaagtcaattcagttggtacctcgagtgactatgaaatgaaaaggtctgtgagtacggttgttcttggacacggggaggaaggattggactccggaccccaaggagtctggtagaggaagtaagacccgtcacgggtcggtggccgactgggtgtggactgtgaaggtcggtcagacgacggacgagggagaaggactttgacctgacaaggacccttttcccacttcggtggagatcttccctgaaaccggaggggggttcttgaagggtaccatcttaccccaccccctcctcccgcgtgcccgactcgcctatccccgccgggcctcggtcggtccgtcaaaataactttagaaaaa

SEQ ID NO: 13 - DNA, 293 bp, Homo sapiens
ggtaaacggacagagacgagaggggcaagggaggacagtggatgacagggaagacgagtgggggcagagctgctcaggaccatggtttttttgtcatcacctatgcagatctgagacccaggggctgatgatgatggggaaatcacctacgagaatgttcaagtgcccgcagtcctaggggagccctcaagcttggcttcttctgtactaggggacaaagcagcggtcaagtcggagcagccaactgcgtcctggagagccgcgacgtcaccagctgtcgggcggattctcgc

SEQ ID NO: 14 - DNA, 288 bp, Homo sapiens
ggtaaacggacagagacgagaggggcaagggaggacagtggatgacagggaagacgagtgggggcagagctgctcaggatcatggctgaggccatcacctatgcagatctgagacccaggggctgatgatgatggggaaatcacctacgagaatgttcaagtgcccgcagtcctaggggtgccctcaagcttggcttcttctgtactaggggacaaagcagcggtcaagtcggagcagccaactgcgtcctggagagccgtgacgtcaccagttgtcgggcggattgt

SEQ ID NO: 15 - DNA, 9655 bp, Homo sapiens
ctattggcagagctctagggtccctggcagtgtcctgccacacaccaccaatcatccgagctgttggcatgtggtgaggaccttttcccccttcctttttcctccccacagctgcaggagggggattctgcggggcggatgtcactttgctaggggcgggttcttcccacccccacactacatcttgttctagctccttcctatcccggatgtaggccattttggggttcctagtaggggtcacatcccggtaaaagaacattagggagcacctttgggcacccctgctcagcagccccctttcccccaactggttccctctctcctccatcagccaggcccccaccacctatgagggcctcgcggtccctgggtcctccgccacaaatgccccctttttccttggggctgggtgggaagggacagctttgggactttttaaagaaagaactttgctaggaaagtaagagacgaaccaaaaccaaaccaagaaaatgtcaagacctccgctggtccggaatctgcgctcggcctctggctctgttctgctcctctgccttgacttcacctaggaagctgcagccttcttaagccctctgcaactcccttgctcacctgctccctggctccccatgagggtgcatggggggcagtgctgccacttgcctttccagaggtgctgcagccatggctcttccttaccttcacctctggcccaggcctgttcctgaacccccagttactggaacccccaggatgatgagaagcgagggatgcaaaccaggaacccccagtctcatggggagaacggagcctcaccctggagctggggagatcgtctgggtcagtgcagctccagctacacatcataagccccagtggacaaggcagccaggaggagggtggggcagatcagggcagataaccagaaggaccgagtttggggcccagcatggcccatatgggtttaagatgcatggagaggaagcacagggaggacatggggacaggcccatcccctacccctgtgttctggacctcagcgtcccctccgtggcccctctggccctggcccagggatgccttctgcttcattgagcccatctgtttgagacttgctaacaccttcctcttttgtgactggaagtcacagggccaattgctaagccgtgcagtcacagagggaacacagagcctagttgtaaacggacagagacgagaggggcaagggaggacagtggatgacagggaagacgagtgggggcagagctgctcaggaccatggtttttttgtcatcacctatgcagatctgaggtttgtgaaggctcccctgaagaagagcatctccagccggttaggacagggtaagggggatgaggcctccccttgatcctgcatccccacttcccgccccacagatcccattcccaaatcccaccttggtcccttttctcttctctctagacccaggggctgatgatgatggggaaatcacctacgagaatgttcaagtgcccgcagtcctaggggtgccctcaagcttggcttcttctgtactaggggacaaagcaggtctggagagcctgggggatgtgtgtttgttggggggctgtgtcttgagctggggagtccacaatgtttttgttgttgttcttgagacagggtcttgttctgtcactcaggctggagtgcagtggtgtgatcatggctcactgcagcctcgacctcctgggctcaagtgatcctcctgccctggcctcccaaagtgttgggattgcaggcgtgcgcccccatgccatggccctcagtattcttgagagcacaataccctggggaaaggaggagggcggttcttaggaaacagaagaataggaacaggataaatgggagccaggggaggaagaaagattcctcccgcaaggtggagacccctttcggggagataagggctgcggagggatagggcagccctgtgggggatgagttctaatgggaaacggggagccatgggagggaaaagaggtgatgggagatggatgagagcaaagggggagagggtttggagccgacagataaggggcctctgaggaaacgataaagggagaactaggagagatgagggggtcccgggaaaagatacagtgagacccaggaggaagccgagagagtcccgggcagaggaggaactcaggagagtgacactgtgtcccagagcagctctagcaggcaacgtagggcagactggagtttcccagtggcgagggcgaagcgggtgcgcaggaggagaaggggcaacactcggggcttccggtctcacactggatgctcccctcgacagcggtcaagtcggagcagccaactgcgtcctggagagccgtgacgtcaccagctgtcgggcggattctcccctgtgagtgtgcctgtgcggggcagccgcggggccaagtgctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggtaacgcccccgagtccccaccagcactgattctggaacaccctctcccctacctgcgaaacaccccgctacccgacagaaaccaccatgcttgccccctcagctctgcaccgccctcctcttagggatgccttccccaggggtctcccccacggacctgtcccttcctctctacattctttacgggggctgactgccctcatccatcccactgtccccctaaggccgcacaacctgcctgcgatacctcctgctcggcctgctcctcacctgcctgctgttaggagtgaccgccatctgcctgggagtgcgctgtgagtaaggctgtccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcaggtatccgctctggggagaggagagaagggagggatggattggagcaaggagatagtagtggcaggagtctatcacccaggggcctgtgaggtgttcaggaaaggggcagtgaaccagagtggtggctgacatgagctgttcagccttggccaacagagagaaggaccaagctgcatactagatttagggggacccacccagctccaaggctcatcctgtaggggagaggaggatggcagccagcagtccaaatcacactgtccaaggtttcttccggttgatatcagtgacaaggatctgggaacgcccccaaatttggatgtgaacttggcggtttgcacatctttctaaagacagggacctcatttttaattaattttcgttatagctgagatctccccaaagggtaagacttactggctgacatggagtgcaacctggtggtggggcaaggcagtgaggcgctcagcactcacagtgaggtggagctgggagttgtggtgtagggaactaggatggggtgtccattcattttgaaagcaggaccccagccagtaagtggagatggggaaagggacacataaaagaggcaaggctgtatcaaaacaggctgagggtctaggcaaggtgccacgcctaaggggactggggtgtttttgggctatgatcaggtttaggctctgtctagcttgggggtcatctgggtgatctccccaggcataagacagcaagattatgtgcagacaccccttgccctcatttccttaaccttaccctctgtgaccatcagttccatgccagggctgctcttagcagcatgttagacctctacctcccaaattgctcttccggcgaaaggatggtttcagacttccactctgatcacagctgcccaccactcctgcaggttttccccacacttttttgatctcatcaaggcctccaatctgctgactcccctttttctcccttcccatcagtccaggcctccaatcaactgattcccctttttctctcttcccatcagcttctcctgtccacacttctctccctacctaacatgggtttggagcccatcacttcaactactcacttatttttttaaatagagacaaggtctcactatgtttccctggccggtatcgaactcctggactcaagcgatcctcctgcctcagcctcccaaagtgctgagattacagtcgtgagccaccgcacctggctcactcacttctttgttccattatccttctagtgtacctgatgggcaaaatttgaactctggatgaacccaactgcccatggtcagcatcaaggtgtgacaaattgtggaatcattctgctcagtgccactgagactctagtctctcagcactgcctggcaacctttgcaatggtcagcttgctctccatagtaactccttctccctcactttcagcagatgtcctcatcttccactctttcagagaggaattcccttcactttccatcaccaagtctatatatccatatggatttttgcccatcctcatcttttgcaaggatatctgcaacagctttctaaatgtttttcctactcactcctctccctgcagtccattcttcatattggagccagggtaatcttttacaaacaaaaatctgatcgtgtcacccaatctacagcattccagttaattgattacataaacagttatggaatacataacatataatatgttctagcactgtgcccaggcacttgggatatagctagtgaacaaaattcctctatgtaaaaccctacactgactttctcttcctcataatataaaaataaaaatccttaacatggcttccatcactcctgcctacctttcagtgtgccacttatctcctcaccctcttccatttggccatacagatttctttttttttttttttgaggcggagtctcgctctgtgagactgaagtgcactcccaggctggagtgcagtggtgtgatctgggctcactgcaacctctgcctcctggattcaagctatccttttgcctcagcctcccgagtaactgggattataggcatgcgccatggtgcctgggtattttttggtatttttagtagaggtggggtttcaccatgttgaccaggctggtcttaaactcctgacttcaggtgatccaccccgcctcggcctcccaaagtgctgggattacagaagtgagccattgtgcccagcagggatttctttttattcctttaagttgccataggacagtagctcaagggagtagcagggtggggacatgagcatatgtagagagaagagagggagctagtggagagggaatgatttttgagtgatgaaggtaggactgatggaacaaagcaatggagagaggtggcctgggatcagaagtgcacattgggatgtaggatgttgaagaaagagggatatttatttcttatggatagtcaggaaaagaagaacagagaagagaaattttgaggtggagaggagggaaaaggagagagctcagtttctgcaaaacagtacacaacattttctgcttgtaggaaattggagagggacggagggggccagtgctggaggagtggaggatgtgcagtgaggggtgctttagggagtggaggagatgggcccaagagatgttaggtaaagtgtcagggtggactgcaattgttcagaaattggggtaataaaatcagccctattaagacagacctcccagattgctaagaagtaatgttgttgaatgttaaagtctttttactaagtaaggacaggtatggtgtttccaggggatgcaaaagaaagattgcaaagatcatagaagtccatgaaggtgctgagattagctctgggggaatattgttagcaaaagattgctgagtagtcactttcatcccaactgttgctgctatcacactctttctgctttttagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcagtaagcatatccttactcacagactgcagtgggtactattaggtaaagggacatatgtcatcaagtcaggaatggcagtgcacacatggctggggtggccattccttttccatggaagaaactgagaatcataatactcttgacctctgagcttggaaacaacctctgcatcttctcagcacagtggtccaggcagcccctagtaactgattagagttggcatgagaatggaaacttaaccatatctacatttttttttttttgagacagagtctcactctgtcacccaggctggagtgcacttgcataatctcagctcactgcaacctccgcctcctgggttcatgcaattctcccgtctcagcctcctgagtagctgggactacagtcacatgccaccacgcccagctaatttttgtatttttaatagagtcggggtttcaccatattggtcaggctggtctctaactcctggcctcaggtgatccacctgcttcagtctcccaaagtgctgggattataggtgtgaaccactgcgcccggcctctactttaatgtgtatctcatctcagcttgctggtcagctccctgggctgcttgacaggttcctggactcttgtggcctctcaggcagagaggtcccacacctggtgggctggattgtagggattagttccttggggccagaacagcacacagaatgactcccctccaaaggctggcagagattcttactcagctggaaggcaaatttagcagtgagcctgcatctgcagggtggcacttgatggattcagggacaagttaccaaagagctgtcatatatacagcccccttgccaaagctgaggcctccctagggtgtgcattttcttccatatttctcatcactctccatgtatctctggtctctctgaatctcccctcagcactcttactacttcttaaattcactgttgccaaatggtggttcagggaattcatattggactggcctcagctctaacaaggattggaagttgactgatgatacacaacgcactaggtaagtttgttaggacttctgggtattttcaactccataatcccctccataaagacatggtaatcagatttccttcccacctggtcccctcaggactctcccataaatcaacactgtaattgcctatttgtctgtttcctctatttgattaataacttcttagggcaatcttattctccattctatttccacctagaagtccctggcacatacttttttgtttcagtaactactaggttgggacctacgctatcttttagggggagacattaaggatttagttaaggctccacagctgatgacagcagcgtttctccatccttcgagaaggccacccaaactgaggttgtactttctcagtagcggccacttggggcactagagctgcctcagtgaagtacgcaagggagcagcgggggtaccatcgaagctccacttcactgtgatcaagacagcagtggggtaccattgaggatcaaccctagggaccctgaaacagggtggcaacctccttatgttcctcaattggccttcccatcttttttttttttttgagatggagtctcgctctgtcgcccagactggagtgcagtggtgcaatctcagctcactgcaagctccgcctcaggggttcacaccattctcctgcctcagcctccccagcagctgggactacaggcgcacaccgccatgcccggctaattttttgtatttttagtagagacggggtttcaccgtgttagtcaggatggtctcgatgtcctgaccttgtgatccacccgccttggcctcccaaagtgctgggattgcaggcatgagccaccgtgcccagcctggccttcccatctttaagatctcgagttatttttccctctgctggttctaaagtctgttttagttgaactccagacatctgtcctgaaccatgcccagatgtggccttaggcaggcagggtgagttaggatttaggttttatacaggggaaggggagagaagggagggtgggatatgtccagagcatatcccagctctaccttactactttttaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttggtaagagtgagggatggggctgaggcatggggagtccaggggcagagggactcagagcagggactcagagcaggggcccaggggcccgagagaaaacttgtacctgggatgaaagttcctccacagcctccagttgcttggtctgcttgttagggtggggtgggcagggagggacaggtaaggcctacaacttatgggaggctggtggtgtggctgtcgagagctgaggaggagatggggtgagtgggatgagggttcagttaagtcaaccatggagctcactgatactttacttttccagacactcatgccaacaagaacctgtgcccctccttcctaacctgaggcctggggttcctcagaccatctccttcattctgggcagtgcccagccaccggctgacccacacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattgcacgtgttagtctcatgtgtcagcaatgctgtgtctggttcagtgat

SEQ ID NO: 16 - protein, 14 aa, Homo sapiens
MAEAITYADLRFVK

SEQ ID NO: 17 - DNA, 613 bp, Homo sapiens
aaattggcaggagagccaaaaacaatgtgaaactctgtcttccaagctggccacattcagtgaaatttatccacaatcacactcttactacttcttaaattcactgttgccaaatggtggttcagggaattcatattggactggcctcagctctaacaaggattggaagttgactgatgatacacaacgcactaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttgacactcatgccaacaagaacctgtgcccctccttcctaacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataataaaaaaaaaaaaaaa

SEQ ID NO: 18 - DNA, 9584 bp, Homo sapiens
ctattggcagagctctagggtccctggcagtgtcctgccacacaccaccaatcatccgagctgttggcatgtggtgaggaccttttcccccttcctttttcctccccacagctgcaggagggggattctgcggggcggatgtcactttgctaggggcgggttcttcccacccccacactacatcttgttctagctccttcctatcccggatgtaggccattttggggttcctagtaggggtcacatcccggtaaaagaacattagggagcacctttgggcacccctgctcagcagccccctttcccccaactggttccctctctcctccatcagccaggcccccaccacctatgagggcctcgcggtccctgggtcctccgccacaaatgccccctttttccttggggctgggtgggaagggacagctttgggactttttaaagaaagaactttgctaggaaagtaagagacgaaccaaaaccaaaccaagaaaatgtcaagacctccgctggtccggaatctgcgctcggcctctggctctgttctgctcctctgccttgacttcacctaggaagctgcagccttcttaagccctctgcaactcccttgctcacctgctccctggctccccatgagggtgcatggggggcagtgctgccacttgcctttccagaggtgctgcagccatggctcttccttaccttcacctctggcccaggcctgttcctgaacccccagttactggaacccccaggatgatgagaagcgagggatgcaaaccaggaacccccagtctcatggggagaacggagcctcaccctggagctggggagatcgtctgggtcagtgcagctccagctacacatcataagccccagtggacaaggcagccaggaggagggtggggcagatcagggcagataaccagaaggaccgagtttggggcccagcatggcccatatgggtttaagatgcatggagaggaagcacagggaggacatggggacaggcccatcccctacccctgtgttctggacctcagcgtcccctccgtggcccctctggccctggcccagggatgccttctgcttcattgagcccatctgtttgagacttgctaacaccttcctcttttgtgactggaagtcacagggccaattgctaagccgtgcagtcacagagggaacacagagcctagttgtaaacggacagagacgagaggggcaagggaggacagtggatgacagggaagacgagtgggggcagagctgctcaggaccatggctgaggccatcacctatgcagatctgaggtttgtgaaggctcccctgaagaagagcatctccagccggttaggacagggtaagggggatgaggcctccccttgatcctgcatccccacttcccgccccacagatcccattcccaaatcccaccttggtcccttttctcttctctctagacccaggggctgatgatgatggggaaatcacctacgagaatgttcaagtgcccgcagtcctaggggtgccctcaagcttggcttcttctgtactaggggacaaagcaggtctggagagcctgggggatgtgtgtttgttggggggctgtgtcttgagctggggagtccacaatgtttttgttgttgttcttgagacagggtcttgttctgtcactcaggctggagtgcagtggtgtgatcatggctcactgcagcctcgacctcctgggctcaagtgatcctcctgccctggcctcccaaagtgttgggattgcaggcgtgcgcccccatgccatggccctcagtattcttgagagcacaataccctggggaaaggaggagggcggttcttaggaaacagaagaataggaacaggataaatgggagccaggggaggaagaaagattcctcccgcaaggtggagacccctttcggggagataagggctgcggagggatagggcagccctgtgggggatgagttctaatgggaaacggggagccatgggagggaaaagaggtgatgggagatggatgagagcaaagggggagagggtttggagccgacagataaggggcctctgaggaaacgataaagggagaactaggagagatgagggggtcccgggaaaagatacagtgagacccaggaggaagccgagagagtcccgggcagaggaggaactcaggagagtgacactgtgtcccagagcagctctagcaggcaacgtagggcagactggagtttcccagtggcgagggcgaagcgggtgcgcaggaggagaaggggcaacactcggggcttccggtctcacactggatgctcccctcgacagcggtcaagtcggagcagccaactgcgtcctggagagccgtgacgtcaccagctgtcgggcggattctcccctgtgagtgtgcctgtgcggggcagccgcggggccaagtgctcgcgcctgtcccggctgcggatggcgctgggccccgcggggtgcagctccttccgggataaacccgggcgcagtccttccgacgcccgggctgccgtgccacattcaccttcatccggtgcgcctgaatccgacgtgcaccgcccccctgcggatggtcccaattcacctacctgccccccttcccctctctccgtctgatatttccttccccttctcccaggtaacgcccccgagtccccaccagcactgattctggaacaccctctcccctacctgcgaaacaccccgctacccgacagaaaccaccatgcttgccccctcagctctgcaccgccctcctcttagggatgccttccccaggggtctcccccacggacctgtcccttcctctctacattctttacgggggctgactgccctcatccatcccactgtccccctaaggccgcacaacctgcctgcgatacctcctgctcggcctgctcctcacctgcctgctgttaggagtgaccgccatctgcctgggagtgcgctgtgagtaaggctgtccctacttcccacttacaccgaacgactttcgtcccgactctcgccccaaagctgatcttcctcagcagggatcatagtcactaccaccacctccttagctttgggtttggtttccagccagggctgttgtcttcctgaattgtcctcacctaatccagtacctccttacgctttagtcagtcaaatcttgtatttcagcctgtgatgcaaaaagttgcaagaagcagaaaaagggctgatgctgcagtctcagggcactagggcagggctggagaagacatccaccaaatctctgtggccaaacagatctgcaggtgtctcagcagctccagcagacgaacagggttctggaagtcactaacagcagcctgaggcagcagctccgcctcaagataacgcagctgggacagagtgcagaggatctgcaggggtccaggagagagctggcgcagagtcaggaagcactacaggtggaacagagggctcatcaggcggccgaagggcagctacaggcctgccaggcagacagacagaagacgaaggagaccttgcaaagtgaggagcaacagaggagggccttggagcagaagctgagcaacatggagaacagactgaagcccttcttcacatgcggctcagcaggtatccgctctggggagaggagagaagggagggatggattggagcaaggagatagtagtggcaggagtctatcacccaggggcctgtgaggtgttcaggaaaggggcagtgaaccagagtggtggctgacatgagctgttcagccttggccaacagagagaaggaccaagctgcatactagatttagggggacccacccagctccaaggctcatcctgtaggggagaggaggatggcagccagcagtccaaatcacactgtccaaggtttcttccggttgatatcagtgacaaggatctgggaacgcccccaaatttggatgtgaacttggcggtttgcacatctttctaaagacagggacctcatttttaattaattttcgttatagctgagatctccccaaagggtaagacttactggctgacatggagtgcaacctggtggtggggcaaggcagtgaggcgctcagcactcacagtgaggtggagctgggagttgtggtgtagggaactaggatggggtgtccattcattttgaaagcaggaccccagccagtaagtggagatggggaaagggacacataaaagaggcaaggctgtatcaaaacaggctgagggtctaggcaaggtgccacgcctaaggggactggggtgtttttgggctatgatcaggtttaggctctgtctagcttgggggtcatctgggtgatctccccaggcataagacagcaagattatgtgcagacaccccttgccctcatttccttaaccttaccctctgtgaccatcagttccatgccagggctgctcttagcagcatgttagacctctacctcccaaattgctcttccggcgaaaggatggtttcagacttccactctgatcacagctgcccaccactcctgcaggttttccccacacttttttgatctcatcaaggcctccaatctgctgactcccctttttctcccttcccatcagtccaggcctccaatcaactgattcccctttttctctcttcccatcagcttctcctgtccacacttctctccctacctaacatgggtttggagcccatcacttcaactactcacttatttttttaaatagagacaaggtctcactatgtttccctggccggtatcgaactcctggactcaagcgatcctcctgcctcagcctcccaaagtgctgagattacagtcgtgagccaccgcacctggctcactcacttctttgttccattatccttctagtgtacctgatgggcaaaatttgaactctggatgaacccaactgcccatggtcagcatcaaggtgtgacaaattgtggaatcattctgctcagtgccactgagactctagtctctcagcactgcctggcaacctttgcaatggtcagcttgctctccatagtaactccttctccctcactttcagcagatgtcctcatcttccactctttcagagaggaattcccttcactttccatcaccaagtctatatatccatatggatttttgcccatcctcatcttttgcaaggatatctgcaacagctttctaaatgtttttcctactcactcctctccctgcagtccattcttcatattggagccagggtaatcttttacaaacaaaaatctgatcgtgtcacccaatctacagcattccagttaattgattacataaacagttatggaatacataacatataatatgttctagcactgtgcccaggcacttgggatatagctagtgaacaaaattcctctatgtaaaaccctacactgactttctcttcctcataatataaaaataaaaatccttaacatggcttccatcactcctgcctacctttcagtgtgccacttatctcctcaccctcttccatttggccatacagatttctttttttttttttttgaggcggagtctcgctctgtgagactgaagtgcactcccaggctggagtgcagtggtgtgatctgggctcactgcaacctctgcctcctggattcaagctatccttttgcctcagcctcccgagtaactgggattataggcatgcgccatggtgcctgggtattttttggtatttttagtagaggtggggtttcaccatgttgaccaggctggtcttaaactcctgacttcaggtgatccaccccgcctcggcctcccaaagtgctgggattacagaagtgagccattgtgcccagcagggatttctttttattcctttaagttgccataggacagtagctcaagggagtagcagggtggggacatgagcatatgtagagagaagagagggagctagtggagagggaatgatttttgagtgatgaaggtaggactgatggaacaaagcaatggagagaggtggcctgggatcagaagtgcacattgggatgtaggatgttgaagaaagagggatatttatttcttatggatagtcaggaaaagaagaacagagaagagaaattttgaggtggagaggagggaaaaggagagagctcagtttctgcaaaacagtacacaacattttctgcttgtaggaaattggagagggacggagggggccagtgctggaggagtggaggatgtgcagtgaggggtgctttagggagtggaggagatgggcccaagagatgttaggtaaagtgtcagggtggactgcaattgttcagaaattggggtaataaaatcagccctattaagacagacctcccagattgctaagaagtaatgttgttgaatgttaaagtctttttactaagtaaggacaggtatggtgtttccaggggatgcaaaagaaagattgcaaagatcatagaagtccatgaaggtgctgagattagctctgggggaatattgttagcaaaagattgctgagtagtcactttcatcccaactgttgctgctatcacactctttctgctttttagacacctgctgtccgtcgggatggataatgcatcagaaaagctgcttttacatctcacttacttcaaaaaattggcaggagagccaaaaacaatgtgaaactctgtcttccaagctggccacattcagtgaaatttatccacaatcagtaagcatatccttactcacagactgcagtgggtactattaggtaaagggacatatgtcatcaagtcaggaatggcagtgcacacatggctggggtggccattccttttccatggaagaaactgagaatcataatactcttgacctctgagcttggaaacaacctctgcatcttctcagcacagtggtccaggcagcccctagtaactgattagagttggcatgagaatggaaacttaaccatatctacatttttttttttttgagacagagtctcactctgtcacccaggctggagtgcacttgcataatctcagctcactgcaacctccgcctcctgggttcatgcaattctcccgtctcagcctcctgagtagctgggactacagtcacatgccaccacgcccagctaatttttgtatttttaatagagtcggggtttcaccatattggtcaggctggtctctaactcctggcctcaggtgatccacctgcttcagtctcccaaagtgctgggattataggtgtgaaccactgcgcccggcctctactttaatgtgtatctcatctcagcttgctggtcagctccctgggctgcttgacaggttcctggactcttgtggcctctcaggcagagaggtcccacacctggtgggctggattgtagggattagttccttggggccagaacagcacacagaatgactcccctccaaaggctggcagagattcttactcagctggaaggcaaatttagcagtgagcctgcatctgcagggtggcacttgatggattcagggacaagttaccaaagagctgtcatatatacagcccccttgccaaagctgaggcctccctagggtgtgcattttcttccatatttctcatcactctccatgtatctctggtctctctgaatctcccctcagcactcttactacttcttaaattcactgttgccaaatggtggttcagggaattcatattggactggcctcagctctaacaaggattggaagttgactgatgatacacaacgcactaggtaagtttgttaggacttctgggtattttcaactccataatcccctccataaagacatggtaatcagatttccttcccacctggtcccctcaggactctcccataaatcaacactgtaattgcctatttgtctgtttcctctatttgattaataacttcttagggcaatcttattctccattctatttccacctagaagtccctggcacatacttttttgtttcagtaactactaggttgggacctacgctatcttttagggggagacattaaggatttagttaaggctccacagctgatgacagcagcgtttctccatccttcgagaaggccacccaaactgaggttgtactttctcagtagcggccacttggggcactagagctgcctcagtgaagtacgcaagggagcagcgggggtaccatcgaagctccacttcactgtgatcaagacagcagtggggtaccattgaggatcaaccctagggaccctgaaacagggtggcaacctccttatgttcctcaattggccttcccatcttttttttttttttgagatggagtctcgctctgtcgcccagactggagtgcagtggtgcaatctcagctcactgcaagctccgcctcaggggttcacaccattctcctgcctcagcctccccagcagctgggactacaggcgcacaccgccatgcccggctaattttttgtatttttagtagagacggggtttcaccgtgttagtcaggatggtctcgatgtcctgaccttgtgatccacccgccttggcctcccaaagtgctgggattgcaggcatgagccaccgtgcccagcctggccttcccatctttaagatctcgagttatttttccctctgctggttctaaagtctgttttagttgaactccagacatctgtcctgaaccatgcccagatgtggccttaggcaggcagggtgagttaggatttaggttttatacaggggaaggggagagaagggagggtgggatatgtccagagcatatcccagctctaccttactactttttaggacttatgctcaaagctcaaaatgtaacaaggtacataaaacttggtcatggtggacactggagtcagagtcatgtagaagttctcttccctacatctgtgagatgacagctttcaggtttccagattaggacagtcctttgcactgagttggtaagagtgagggatggggctgaggcatggggagtccaggggcagagggactcagagcagggactcagagcaggggcccaggggcccgagagaaaacttgtacctgggatgaaagttcctccacagcctccagttgcttggtctgcttgttagggtggggtgggcagggagggacaggtaaggcctacaacttatgggaggctggtggtgtggctgtcgagagctgaggaggagatggggtgagtgggatgagggttcagttaagtcaaccatggagctcactgatactttacttttccagacactcatgccaacaagaacctgtgcccctccttcctaacctgacacttccagccagtctgctgcctgctccctcttcctgaaactggactgttcctgggaaaagggtgaagccacctctagaagggactttggcctccccccaagaacttcccatggtagaatggggtgggggaggagggcgcacgggctgagcggataggggcggcccggagccagccaggcagttttattgaaatctttttaaataattgcacgtgttagtctcatgtgtcagcaatgctgtgtctggttcagtgat

SEQ ID NO: 19 - DNA, 22 bp, primer
atcagggcagataaccagaagg

SEQ ID NO: 20 - DNA, 21 bp, primer
aaacattgtggactccccagc

SEQ ID NO: 21 - DNA, 1357 bp, Mus sp.
tagctggaagactgtgaagcagaggcgcccagggctatggctgacgctatcacgtatgcagacctgcgctttgtgaaagtgcccctgaagaacagcgcatctaaccatctaggacaggactgtgaggcctatgaagatggggaactcacctacgagaacgtgcaagtgtctccagtcccaggagggccaccaggcttggcttcccctgcactagcggacaaagcaggtcctacagtctgcttgcaatacttcttgcttggccttctcgtgtcctgtctgatgttaggggtggctgtcatctgcctgggagttcgctatctgcaggtgtctcggcagttccaggaggggaccaggatttgggaagccaccaatagcagcctgcagcagcagctcagggagaagataagtcagctggggcagaaggaggtggagcttcagaaggctcggaaagagctgatctcgagccaggacacattacaggagaagcagaggactcacgaggacgctgagcagcaactacaagcctgccaggctgagagagcgaagaccaaggagaacctgaaaactgaggaggagcggaggagggacctggaccagaggttgacaagcacgcgggagacactgaggcgcttcttctctgattcatcagacacctgctgtccatgcggatggattccatatcaggaaaggtgcttttacatctcacataccctcggaagtctggaggagagccaaaaatactgcacatctctgtcctccaaactggcagcattcgatgaaccttctaagcattactatgaatacctctctgacgccccccaggtttctctgcccagcggcttagaggagttgctagatcgttcgaagttatattggatacagatgagcaagaagtggaggcaggactctgactctcaaagccgacattgtgtcaggataaaaacatattaccagaagtgggaaagaacaatttccaagtgtgcagagcttcacccctgcatttgtgagtcggaggctttcaggtttcctgatgggatcaatctgaactgaaacggacacttgaacaagaccttgtgacctacatccttaacctacggcctgccaatttttaagactgctattcctccagcactccctcactctcgggcatgcccagctaagggatgacctgctgcttgcttgaaagctgctccagaaactggacttctcttgggaagagtaaagaagcctccagaaaagacttgaccttccttaagaacttcccaaactagagatgggtcaggggagggcgctccgctgagtggatggatggcccggagccagccaggcagttttattgaaatatttttaaatactt

SEQ ID NO: 22 - protein, 337 aa, Mus sp.
MADAITYADLRFVKVPLKNSASNHLGQDCEAYEDGELTYENVQVSPVPGGPPGLASPALADKAGPTVCLQYFLLGLLVSCLMLGVAVICLGVRYLQVSRQFQEGTRIWEATNSSLQQQLREKISQLGQKEVELQKARKELISSQDTLQEKQRTHEDAEQQLQACQAERAKTKENLKTEEERRRDLDQRLTSTRETLRRFFSDSSDTCCPCGWIPYQERCFYISHTLGSLEESQKYCTSLSSKLAAFDEPSKHYYEYLSDAPQVSLPSGLEELLDRSKLYWIQMSKKWRQDSDSQSRHCVRIKTYYQKWERTISKCAELHPCICESEAFRFPDGINLN

SEQ ID NO: 23 - DNA, 18 bp, primer
agaggcgcccagggctat

SEQ ID NO: 24 - DNA, 21 bp, primer
ccctcccctgacccatctcta

SEQ ID NO: 25 - DNA, 19 bp, primer
agcatatcccagctctacc

SEQ ID NO: 26 - DNA, 20 bp, primer
tgacacatgagactaacacg

(19) United States
(12) Patent Application Publication
(10) Pub. No.: US 2003/0077644 A1
(43) Pub. Date: Apr. 24, 2003

(54) Diagnosis and treatment of diseases caused by mutations in CD72

(76) Inventor: Bing Yang, Saginaw, MI (US)

Correspondence Address: BROOKS & KUSHMAN, 1000 TOWN CENTER 22ND FL, SOUTHFIELD, MI, 48075

(21) Appl. No.: 10/259,042

(22) Filed: Sep. 27, 2002

Related U.S. Application Data
(60) Provisional application No. 60/325,946, filed on Sep. 28, 2001.

(51) Int. Cl. C12Q 1/68; C12P 19/34

(52) U.S. Cl. 435/6; 435/91.2

(57) ABSTRACT

Mutants of the CD72 gene and their protein products are disclosed. Methods of diagnosing various diseases such as thyroid follicular carcinoma, renal cancer, endometrial adenocarcinoma, leukemia, ovarian cancer, lymphoma and lupus erythematosus by detecting particular mutations in human CD72 are also disclosed. Methods for supplying wild-type to cells which have lost normal CD72 function is additionally disclosed. CD72 associated diseases are additionally disclosed.

### CROSS-REFERENCE TO RELATED APPLICATION

This application claims the benefit of U.S. provisional application Serial No. 60/325,946, filed Sep. 28, 2001.

### BACKGROUND OF THE INVENTION

1. Field of the Invention

The present invention relates to diseases resulting from hyperproliferation or hyperactivity of cells. In human thyroid follicular carcinoma, leukemia, renal cancer, endometrial adenocarcinoma, ovarian cancer, chondrosarcoma, lymphoma and human lupus erythematosus, expression of mutated forms of CD72 is described.

2. Background Art

CD72 is a type II membrane glycoprotein with its N-terminus on the cytoplasmic side of the membrane. (For review, see Parnes and Pan, Immunol. Rev. August 2000; 176:75-85, 2000). CD72 has been found to be expressed by a number of cell types including B cells, T cells, vascular smooth muscle cells and a number of tumor cells (Robinson et. al., Immunogenetics. 1997;45(3):195-200, Hammer R. D., et al., Am. J. Surg. Pathol. May 1996;20(5):613-26, Garand R., et al, Leuk. Res. August 1994;18(8):651-2, Gagro A., et al., Leuk. Lymphoma. April 1997; 25(3-4):301-11., Hishima T., et al., Am. J. Pathol. August 1994;145(2):268-75, Mechtersheimer G., et al., Pathol Res Pract. August 1990;186(4):427-38., Schwarting et al., Am. J. Hematol. November 1992;41(3):151-8.)

Four different alleles have been identified for CD72 in mice. Sequence analysis of CD72 between mouse strains revealed that the cytoplasmic domain of CD72 is highly conserved while the extracellular domain is highly polymorphic, especially at the membrane-distal portion of the protein (Robinson et al., supra).

CD72 was initially implicated in B cell regulation, suggesting a role in the immune system. Ligation of CD72 with antibody leads to B cell proliferation and the phosphorylation of the Src kinases Lyn and Blk (Venkataraman C et. al, Eur. J. Immunol. October 1998;28(10):3003-16.). These Src kinase family members are also phosphorylated during B cell activation through the B cell receptor. However, CD72 has been found to be expressed in many other tissues as well. This expression in other tissues suggests that CD72 may have roles unrelated to immune regulation.

CD72 has two immunoreceptor tyrosine-based inhibitory motifs (ITIMs). The first ITIM has been demonstrated to bind to SHP-1 phosphatase (Adachi T. et al., J. Immunol. May 15, 1998;160(10):4662-5). Presumably, SHP-1 can dephosphorylate phosphorylated tyrosines in B cell activation and other cell activation events (Adachi T, et. al., J. Immunol. Feb. 1, 2000;164(3):1223-9). The second ITIM has been shown to bind to Grb2, not SHP-1 (Wu Y., et., al., Curr. Biol. Sep. 10, 1998;8(18):1009-17). Grb2 binds to Sos, a guanine nucleotide exchange factor for Ras (Li N., et al., Nature May 6, 1993;363(6424):85-8., Egan S. E., et al., Nature May 6, 1993;363(6424):45-51). The Ras pathway is involved in cell proliferation and differentiation (for review, see Lodish et. al., Molecular Cell Biology, Freeman and Company 2000).

The Class IV semaphorin CD100 has been shown to be the receptor for CD72 (Kumanogoh A., et al., Immunity November 2000;13(5):621-31). Semaphorins have been implicated in neuron development, organogenesis, and cancer. CD100 expression has also been demonstrated in a number of tissues, including, neural tissues, kidney, lung, and heart (Hall K. T., et al., Proc. Natl. Acad. Sci. USA. Oct. 15, 1996;93(21):11780-5). Additionally, CD100 is the only semaphorin reported to be expressed in T cells (Shi W, et al., Immunity November 2000;13(5):633-42.). Soluble CD100 was also shown to be released during T cell activation and in an animal model for lupus (Delaire S, et al., J. Immunol. Apr. 1, 2001;166(7):4348-54., Wang X., et al., Blood. Jun. 1, 2001;97(11):3498-504).

Many diseases are either the result of the hyperpoliferation or hyperactivity of cells. Cancers result from the hyperpoliferation of otherwise normal cells. Several infectious diseases such as Epstein-Barr, HPV and CMV also cause the hyperproliferation of cells as a result of infection. Autoimmune diseases such as lupus erythematosus and allergies result in a hyperactivation of T cells or B cells.

Many cell surface molecules have been implicated in the regulation of cell activation and cell growth. Cells are subject to both positive cell regulation, such as activation or growth stimulation by hormones, and negative regulation, such as inhibition of cell growth by extracellular ligands. Negative regulation through cell surface molecules plays an important role in regulating cell activity and migration (see review by Ravetch and Lanier, Nature October 2000; 290: 84-89).

If cell growth and activation go unregulated, diseases such as cancer occur. Recently, more and more data support CD72 as a negative regulator of B cell activation. (Pan C., et.al., Immunity. October 1999;11(4):495-506, Adachi T., et al., J. Immunol. May 15, 1998;160(10):4662-5., Adachi T., et al, J. Immunol. Feb. 1, 2000;164(3):1223-9.). CD72 mutations, however, have never been implicated in any diseases characterized by the hyperactivation or hyperpoliferation of cells.

Identification of CD72 mutations found in various diseases would not only offer a strategy for prospectively identifying and treating at risk patients based on their genotype, it would also provide important insights into the molecular mechanisms of this genetic polymorphism.

### SUMMARY OF THE INVENTION

The present invention is based on the observation that a CD72 gene containing mutations and encoding a corresponding mutant protein may be correlated with the presence of diseases such as cancer, systemic lupus erythematosus, allergy, autoimmune disease, Epstein-Barr virus infection, cytomegalovirus infection and papillomavirus infection.

According to the present invention, there is provided a method for the diagnosis of diseases associated with a CD72 mutation in an individual, comprising detecting a mutation in the human CD72 gene, its CD72 mRNA, or in the CD72 protein, wherein the presence of the mutation is indicative of the disease.

It is another object of the invention to provide antibodies that bind to mutant CD72 proteins.

It is another object of the invention to provide primers for detecting and amplifying a region of DNA which contains the CD72 mutations.

It is another object of the invention to provide probes for detecting a region of DNA which contains the CD72 mutations.

It is also an object of the present invention to provide a mutant CD72 gene or an expressed mutant CD72 protein for drug development, gene therapy and other uses to prevent or ameliorate the effects of or resulting from the mutant CD72 gene.

### BRIEF DESCRIPTION OF THE DRAWINGS

FIG. 1 depicts the sources of cDNA libraries which were used to screen for CD72 mutations.

FIG. 2 depicts RT-PCR of CD72 mRNA isolated from NZM mice demonstrating that NZM mice only express the B2 form of CD72. mRNA from the spleens of NZM mice were purified using RNeasy Midi Kits (Qiagen Inc., Valencia, Calif.). A set of primers were designed with the following sequences: 5′ primer AGAGGCGCCCAGGGCTAT (SEQ. ID. NO:23); 3′ primer CCCTCCCCTGACCCATCTCTA (SEQ. ID. NO:24). First round of RT-PCR was conducted using Titan One Tube RT-PCR (Roche Diagnostics GmbH, Germany). A second round of PCR utilized PCR Core System Kits from Promega Co. (Madison, Wis.). Annealing temperature set at 60° C., Mg++ concentration was at 1.5 mM. The products were analyzed in 1% agarose gel. Lane 1 is the PCR product from NZM mice. Lane 2 is the Φ X 174 Hae III fragment with molecular sizes at 1353 bp, 1078 bp, 873 bp, 603 bp.

FIG. 3 depicts a Western blotting analysis of the tissue homogenates from endometrial carcinoma tissue (Lane 1) and normal surrounding tissue (Lane 2) from the same patient. The homogenates were prepared with lysis buffer containing SDS. The samples were subjected to 10% SDS-PAGE gel under reducing conditions. After electrophoresis, the proteins were transferred to nitrocellulose membrane. The membrane was blocked with 3% non-fat milk from Pierce Co (Rockford, Ill.). Polyclonal rabbit anti-CD72 (Santa Cruz Biotech) was used as the first antibody for blotting of 60 minutes at 25° C. The membranes were washed 5 times with phosphate buffered saline containing 0.05% Tween-20. HRP-conjugated goat anti-rabbit antibody (Pierce) was then added as the second antibody. The membranes were then washed 5 times with phosphate buffered saline containing 0.05% Tween-20. West femto chemiluminescence kits from Pierce Co. (Rockford, Ill.) were used to develop the bands as shown according to the directions on the kit. Protein loading for Lane 2 is 50% higher than Lane 1 as determined by BCA kit from Pierce Co. (Rockford, Ill.).

FIG. 4 depicts a Western blotting analysis of tissue homogenates from renal tumors (Lane A2, Lane A4, Lane B2, Lane B4 and Lane B6) and normal surrounding tissues (Lane A1, Lane A3, Lane B 1, Lane B3 and Lane B5) from the same patients. These tissue samples were prepared with lysis buffer containing NP-40. The samples were subjected to 10% SDS-PAGE gel under reducing conditions. After electrophoresis, the proteins were transferred to nitrocellulose membrane. The membrane was blocked with Superblock from Pierce Co. (Rockford, Ill.). Polyclonal rabbit anti-CD72 (Santa Cruz Biotech, Santa Cruz, Calif.) was used as the first antibody for blotting of 60 minutes at 25° C. The membranes were washed 5 times with phosphate buffered saline containing 0.05% Tween-20. HRP-conjugated goat anti-rabbit antibody (Pierce) was then added as the secondary antibody. The membranes were then washed 5 times with phosphate buffered saline containing 0.05% Tween-20. West femto chemiluminescence kits from Pierce Co. (Rockford, Ill.) were used to develop the bands as shown according to the directions on the kit.

FIG. 5 depicts a Western blotting analysis of CD72 in endometrial carcinoma tissues. Tissue homogenates from endometrial carcinoma tissues (Lane 2, Lane 4, and Lane 5) and normal surrounding tissues (Lane 1 and Lane 3) were prepared with lysis buffer containing NP-40. The Western blotting procedures of FIG. 4 were used to detect CD72.

FIG. 6 depicts a Western blotting analysis of cell lysates from ECC-1 cells (Lane 5), and tissue homogenates from ovarian tumor tissues (Lane 2 and Lane 3) and normal surrounding tissues (Lane 1 and Lane 4) from the same patients. These samples were prepared with lysis buffer containing NP-40. The samples were subjected to 10% SDS-PAGE gel under reducing conditions. After electrophoresis, the proteins were transferred to nitrocellulose membrane. The membrane were blocked with 3% non-fat milk. Polyclonal rabbit anti-CD72 (Santa Cruz Biotech, Santa Cruz, Calif.) was used as the first antibody for blotting of 60 minutes at 25° C. The membranes were washed 5 times with phosphate buffered saline containing 0.05% Tween-20. HRP-conjugated goat anti-rabbit antibody (Pierce Co., Rockford, Ill.) was then added as the secondary antibody. The membranes were then washed 5 times with phosphate buffered saline containing 0.05% Tween-20. West femto chemiluminescence kits from Pierce Co. (Rockford, Ill.) were used to develop the bands as shown according to the directions on the kit.

FIG. 7 depicts a PCR analysis of the CD72 gene of ECC-1 Cells and a tumor sample. Genomic DNA from the ECC-1 cell line (Lane 2 and Lane 4) and a tumor sample (Lane 3) were analyzed by PCR using two sets of primers; ITIM primers (SEQ. ID. NO.: 19 and 20) (Lane 2) and EXON89 primers (SEQ. ID. NO.: 25 and 26) (Lane 3 and Lane 4). The PCR products were subjected to 1% agarose gel electrophoresis. Lane 1 is the 100 bp PCR marker. The PCR product from a normal CD72 gene would have 729 nucleotides for the ITIM primers or 830 nucleotides for the EXON89 primers. If there is mutation in the primer regions, there will be no amplification. If there is deletion or insertion between the primers, the PCR product will have a shorter or larger size.

FIG. 8 depicts a Western blotting analysis of tissue homogenates from lymphomas (Lane 1-3) and endometrial carcinoma tissues (Lane 4 and Lane 5). Homogenates were prepared with lysis buffer containing SDS. Equal amounts of protein were loaded in each lane as determined by BCA protein kit from Pierce. The same procedures used to detect CD72 in FIG. 3 were employed. Lane 1 is a sample from tumor tissue of Hodgkin's lymphoma. Lane 2 is a sample of lymphoma. Lane 3 is a sample from a B cell lymphoma of large cell type. Lane 4 and Lane 5 are samples from endometrial carcinoma.

FIG. 9 depicts a Western blotting analysis of protein homogenates from spleen of NZM and C57BL6/J mice. Equal amounts of protein lysates were analyzed by Western blotting as in FIG. 3. Lane M contains homogenates from NZM mice. Lane C contains homogenates from C57BL/6J mice. Panel B represents a shorter exposure of a membrane with less efficiency of transfer than in Panel A.

FIG. 10 depicts a Western blotting analysis of protein homogenates from the spleen of NZM and C57BL6/J mice. Equal amounts of protein lysates from spleen of NZM (M) and C57BL6/J (C) were analyzed by Western blotting as in FIG. 3 except that the first antibody was a monoclonal antibody against CD72 from Santa Cruz Biotech Inc. (Santa Cruz, Calif.) and a HRP-conjugated goat anti-mouse antibody from Pierce Co. (Rockford, Ill.) was used as the secondary antibody. Lane M contains homogenates from NZM mice. Lane C contains homogenates from C57BL/6J mice.

FIG. 11 depicts the DNA sequencing profile of a patient with severe lupus. Blood was drawn from a patient with severe lupus. The patient has symptoms in brain, kidney and joints. Leukocytes were obtained by Ficoll-Hypaque centrifugation. Genomic DNA was prepared using the Qiagen DNeasy Kit according to manufacturer's instructions(Valencia, Calif.). ITIM primers (SEQ. ID. NOS.: 19 and 20) were used in the PCR amplification. The PCR products were run on 1% agarose gel for 1 hour. The DNA band was cut out from the gel and purified using Qiagen gel kit (Valencia, Calif.). Purified DNA was sent to University of Michigan Sequence Core for sequencing using ITIM forward primer (SEQ. ID. NO. 19) as the sequencing primer. The region that has mutations in the lupus patient is shown.

### DETAILED DESCRIPTION OF THE PREFERRED EMBODIMENTS

Four alleles of the mouse CD72 sequences are available in Genbank. Analysis of the CD72 locus of house mouse reveals polymorphism in CD72 between common inbred mice strains. However, the cytoplasmic domain of mouse CD72 is highly conserved between these strains, particularly in the two ITIM domains. The cytoplasmic domain of CD72 is also highly conserved between humans and mice. The normal human CD72 gene sequence has been reported by the Human Genome Project (SEQ. ID. NO:1). When the normal human CD72 gene was analyzed, 9 exons were found to code for CD72 protein, which is similar to mouse CD72 gene.

Mutant CD72 sequences can be identified by screening sequences deposited and available in public databases such as GenBank or other private sequence databases. Sequence identity (at either the amino acid or nucleotide level) within defined regions of the molecule or across the full-length sequence can be determined through sequence alignment using computer software programs such as BLAST, BLAST-2, ALIGN, DNAstar, and INHERIT which employ various algorithms to measure homology.

The BLAST approach, as detailed in Karlin et al., Proc. Nat'l. Acad. Sci. USA 90:5873-5787 (1993), and incorporated herein by reference, searched for matches between a query sequence and a database sequence. BLAST evaluated the statistical significance of any matches found, and reported only those matches that satisfy the user-selected threshold of significance. In this application, the threshold was set at 10 for nucleotide searches.

A search of Genbank revealed more than 100 sequences or expressed sequence tags (EST) labeled as human CD72. The majority of these sequences came from the Integrated Molecular Analysis of Genomes and their Expression (“IMAGE”) Consortium clones (Lennon, G. G., et., al., Genomics 33:151-152 1996). Forty of these clones were obtained from the IMAGE consortium, distributed by Incyte Genomics Inc. (St. Louis, Mo.). These clones had been partially sequenced and all were claimed to match the human CD72 gene.

The IMAGE cDNA clones obtained from Incyte Genomics are in tranformed E. coli. Bacteria containing the cDNA from these clones was streaked onto LB plates with ampicillin and incubated overnight at 37° C. A well-isolated clone was then inoculated into 10 ml of LB broth containing ampicillin and cultured in a shaker overnight at 37° C. Plasmids were prepared using Qiagen mini-prep Kits (Qiagen Inc., Valencia, Calif.) as indicated by the manufacturers directions. Purified plasmids were sent to University of Michigan DNA Sequencing Core for DNA sequencing by Sanger's method, which is well known in the art.

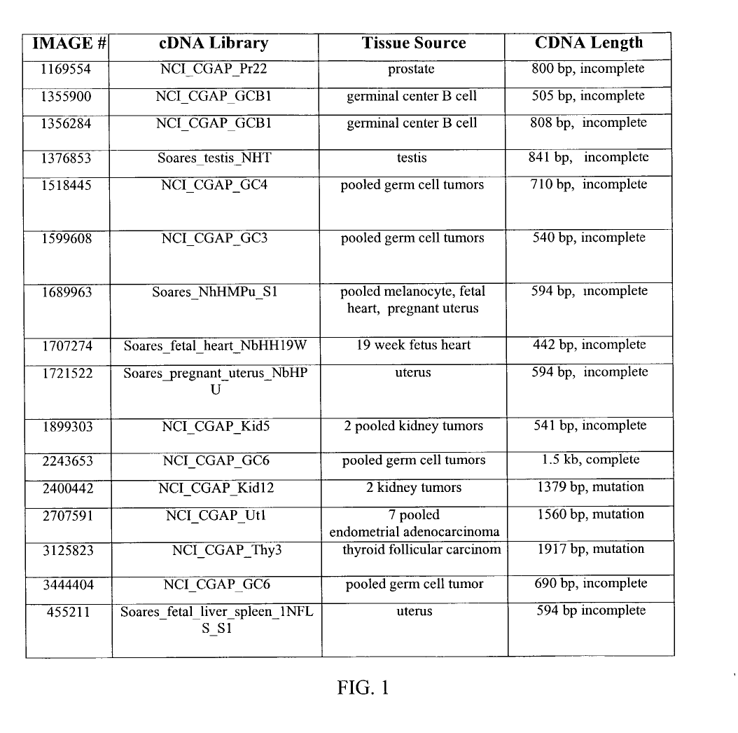

The DNA sequences were compared to the sequences present in Genbank. Almost half of the clones matched to the human testis specific kinase 1, not CD72. All the clones that contained the human CD72 gene showed highly conserved nucleotide sequences. Thirteen clones from different cDNA libraries have shown almost 100% conservation in nucleotide sequence. The cDNA libraries were prepared from 17 different human pools. (FIG. 1).

By alignment and comparison of the clone's DNA sequences with the normal CD72 cDNA sequence, a cDNA clone from thyroid follicular carcinoma tissue was found to have a mutation in the cytoplasmic domain of human CD72 (SEQ. ID. NO:5). This mutation is the result of a truncation in the cDNA. A new segment of cDNA has replaced the segment of human C72 cDNA coding for the first ITIM domain in human CD72 protein. Codons 1 to 189 are deleted from the normal CD72 cDNA. The wild-type CD72 cDNA sequence is depicted in SEQ. ID. NO:2. An insertion of 452 base pairs at this site results in the mutant CD72 cDNA sequence. (SEQ. ID. NO:5.) These mutations result in a mutant CD72 protein with the sequence of SEQ. ID. NO:6. Without the ITIM domain, this region of CD72 can not be phosphorylated and thus, can not recruit phosphatase SHP-1. Therefore, the mutated CD72 can not function properly as a negative regulator because the recruitment of phosphatase SHP-1 is responsible for dephosphorylation events in signal transduction.

A BLAST of the mutant cDNA sequence of SEQ. ID. NO:5 against Chromosome 9 (Genbank Accession No. AL357874) reveals a deletion between codons 843 and 1462 of the genomic CD72 sequence (SEQ. ID. NO:1) which includes Exon 1, resulting in the intronic sequence of codons 716 to 842 being transcribed into mRNA. The new sequence at the 5′ end of the mutated genomic sequence matches Chromosome 9 (AL357874) codons 54511 to 54950, while the rest of the sequence starts at codon 26973 of AL357874. This suggests a 27,537 base pair gap in the genomic sequence. This suggests an additional deletion or translocation of chromosomal DNA in the CD72 gene region, resulting in the mutated genomic CD72 sequence of SEQ. ID. NO:4.

In a cDNA clone from renal cancer, also obtained from the IMAGE consortium, an insertion of 225 bp was found which included part of the intron between Exon 3 and Exon 4, an intron which should have been spliced during mRNA maturation. (SEQ. ID. NO:7). This mutation results in the mutated CD72 protein sequence of SEQ. ID. NO:8. This insertion is in the cytoplasmic domain. As the cDNA in this clone is not long enough, it is uncertain whether the ITIM domains are intact. However, the insertion introduces a stop codon at codon 137 of SEQ. ID. NO:7, thus no mature protein can be translated.

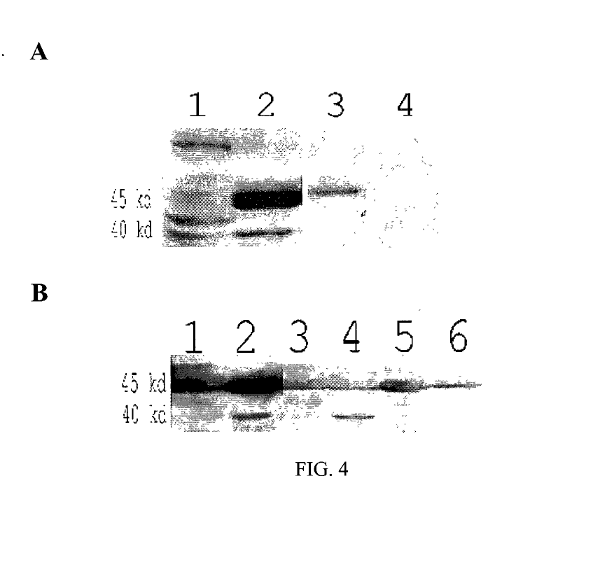

Western blotting analysis of 5 renal cancer tumor samples revealed an increased expression of CD72 in one tumor sample when compared to the normal surrounding tissue. (FIG. 4A, Lane 2). In another tumor sample, a decreased expression of CD72 was observed (FIG. 4 A, Lane 4). In addition, a new 40 kd protein band was expressed or significantly increased, similar to the 40 kd protein band observed in endometrial adenocarcinoma and ovarian cancer described below (FIG. 4). Most likely, this 40 kd protein is a truncated CD72.

Several CD72 mutations have also been found in cDNA clones obtained from a pool of seven endometrial adenocarinoma cDNAs. The intron between Exon 4 and Exon 5 (codons 2986-3306 of SEQ. ID. NO:1) is not processed. This intron contains two stop codons (located at codons 237 and 243 of SEQ. ID. NO. 10) that are in-frame with the normal CD72 open reading frame. Thus a mature CD72 protein can not be translated. In addition, a deletion of 61 bp between codons 1213-1273 of the normal CD 72 cDNA (SEQ. ID. NO:2) was found. The deletion is not in the open reading frame. In addition, there is a point mutation (A to T) at codon 233. These mutations result in a mutated CD72 cDNA (SEQ. ID. NO:10). The mutated genomic sequence has the sequence of SEQ. ID. NO:9.

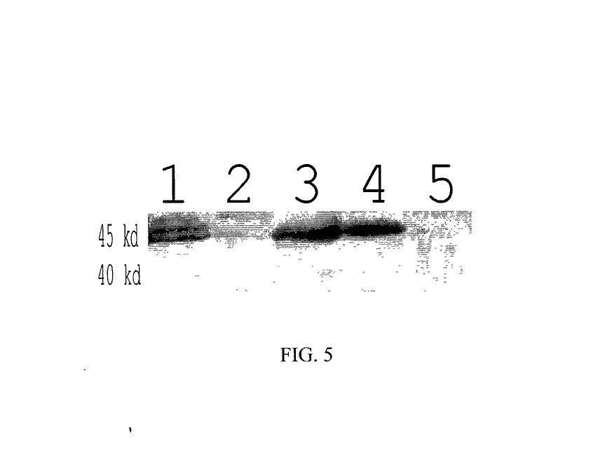

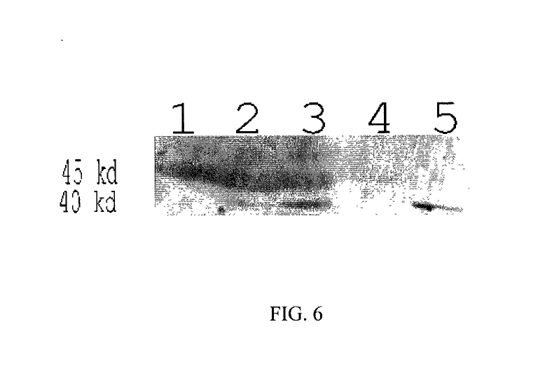

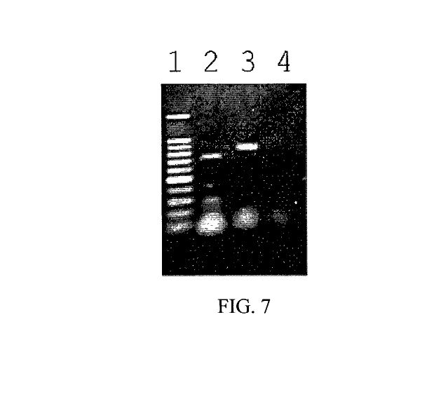

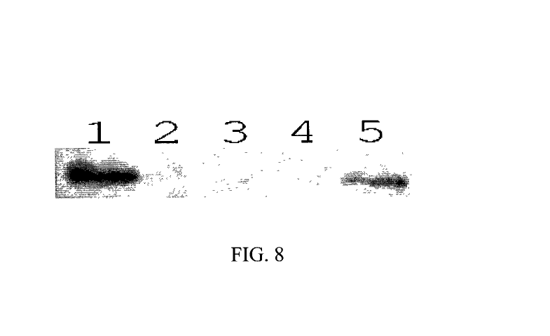

Western blotting analysis of an endometrial adenocarcinoma tumor revealed that normal CD72 protein was not expressed. In one tumor sample, no CD72 was expressed (FIG. 8 Lane 4). In another tumor sample, a lower molecular weight 40 kd protein was detected at a significantly higher amount.(FIG. 5, Lane 5). In addition, an endometrial adenocarcinoma cell line (ECC-1) also expressed a CD72 protein with a molecular weight of 40 kd, lower than the normal molecular weight of 45 kd. (FIG. 6, lane 5). In order to analyze the mutation in the ECC-1 cell line, primers were designed to amplify the first two exons (ITIM primers, SEQ. ID. NOs.: 19 and 20) and the last two exons (EXON89 primers; SEQ. ID. NOs.: 25 and 26) of the CD72 gene. (FIG. 7). PCR analysis using the ITIM primers (SEQ. ID. Nos.: 19 and 20) revealed a band at the predicted size. (FIG. 7, Lane 2). However, when the EXON89 primer was used, a product at the predicted size was not detected (FIG. 7, Lane 4) although the positive control gave a band at the predicted size (FIG. 7, Lane 3). This result has been achieved in numerous experiments. Thus, the EXON89 primer is useful in identifying mutations in the CD72 gene.

Similarly, Western Blotting analysis of two ovarian cancer tumor samples demonstrated the expression of a 40 kd protein while there is no expression of this protein in normal surrounding tissue from the same patient. (FIG. 6). Western Blotting analysis of tumors from B-Cell lymphoma and lymphoma reveal an absence of any CD 72 protein. (FIG. 8, Lanes 2 and 3).

A BLAST search of Genbank also revealed four additional mutant CD72 sequences from pre-B cell leukemia (acute lymphocytic leukemia). These sequences are from clones with Genbank Accession Nos. AF283777, BE243553, BM193456 and BE244747). Three of these sequences: AF283777 (SEQ. ID. NO.: 11), BM193456 (SEQ. ID. NO.: 13) and BE244747 (SEQ. ID. NO.: 14) have a complete 5′ sequence. These three clones have a deletion of codons 141-190 of the normal CD 72 cDNA (SEQ. ID. NO.: 2). This deletion is most likely the result of alternative splicing (the deleted fragment begins with the sequence GT.) The deletion of codons 141-190 results in a frame shifting mutation and the generation of two consecutive stop codons. Thus, no protein product is produced. The other clone, BE243553 (SEQ. ID. NO.: 12), only contains the 3′ sequence, thus one cannot determine if codons 141-190 have been deleted. However, this clone revealed a mutation in which Intron 8 was transcribed. In addition to the deletion of codons 141-190, BM 193456 also has a serial of 8 thymidines inserted after the start codon, resulting in a frame-shifting mutation. The predicted genomic sequence for BM 193456 is shown in SEQ. ID. NO.: 15.

A cDNA sequence from chondrosarcoma was analyzed. A mutant cDNA CD72 sequence was identified (SEQ. ID. NO.:17). Codons 1255-1323 of the normal CD72 cDNA were deleted, resulting in the sequence of SEQ. ID. NO.: 17. This deletion of 69 bp also occurs in the 3′ nontranslated region of the gene but at a different location than the deletion identified in the cDNA from endometrial adenocarcinoma as described above. The genomic sequence of the mutant chondrosarcoma CD 72 gene is shown in SEQ. ID. NO.: 18.

DNA encoding CD72 mutants can be obtained from any cDNA libraries prepared from tissue believed to possess the mutant gene and to express it at a detectable level. Accordingly, human CD72 mutants can be conveniently obtained from a cDNA library prepared from human tissue, such as described in Sambrook et al., Molecular Cloning: A Laboratory Manual (New York: Cold Spring Harbor Laboratory Press, 1989). Mutant CD72 genes may also be obtained from a genomic library.

Libraries can be screened with probes (such as antibodies to a mutant CD72 sequence) designed to identify the gene of interest or the protein encoded by it. Screening the cDNA or genomic library with the selected probe may be conducted using standard procedures, such as described in Sambrook et al., supra. An alternative means to isolate CD72 mutant genes is to use PCR methodology [Sambrook et al., supra; Dieffenbach et al., PCR Primer: A Laboratory Manual (Cold Spring Harbor Laboratory Press, 1995)].

When screening a cDNA library, the oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like .sup.32 P-labeled ATP, biotinylation or enzyme labeling. Hybridization conditions, including moderate stringency and high stringency, are provided in Sambrook et al., supra.

The polymorphic expression of CD72 in mouse strains not only results from the expression of different alleles in different mouse strains, but also results from the differential mRNA splicing in the same mouse strain. Allele b of CD72 in Balb/c, C57BL/6 has three differentially spliced mRNA forms. One form with the complete sequence is expressed on the cell surface (B1 form). Another form does not contain exon 3 and exon 4 nucleotides including the trans-membrane domain (coded by Exon 4). Thus this form is not expressed on the cell surface (B3 form). One other form does not contain exon 3, which encodes a portion of the cytoplasmic domain. This form still contains two ITIM domains and is expressed on the cell surface (B2 form).

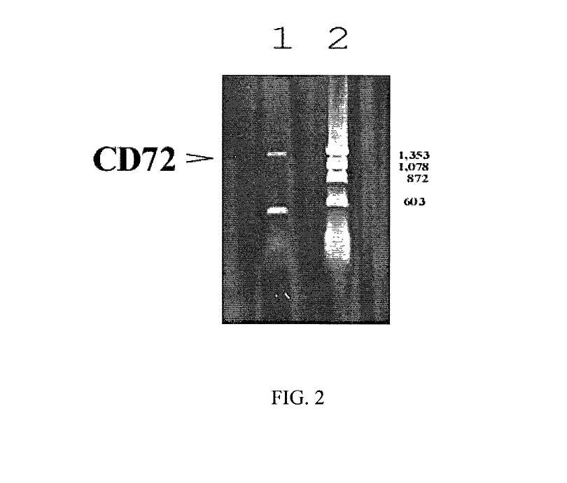

The NZM mouse strain is an animal model for human lupus erythematosus. The mRNA expression of these CD72 forms was analyzed in this mouse strain. Spleen mRNA from NZM mice was purified using RNeasy Midi Kits (Qiagen Inc., Valencia, Calif.). Primers were designed with the following sequences: 5′ primer AGAGGCGCCCAGGGCTAT (SEQ. ID. NO:23); 3′ primer CCCTCCCCTGACCCATCTCTA (SEQ. ID. NO:24). A first cycle of RT-PCR was conducted using Titan One Tube RT-PCR (Roche Diagnostics GmbH, Germany). A second cycle of PCR was then conducted with the same set of primers used PCR Core System Kits from Promega Co. (Madison, Wis.). Three bands corresponded to the size of CD72 mRNA were predicted for CD72 allele b (1.32 for full length DNA, 1.25 for exon 3 deleted DNA, 1.16 kb for exon 3 and exon 4 deleted DNA). In agarose gel analysis, unexpectedly, only one band was observed for NZM mice (FIG. 2). Thus, only one form of mRNA is expressed. This DNA fragment was separated by agarose gel and excised from the gel, centrifuged through a GenElute PCR DNA purification column (Sigma Co., St. Louis, Mo.), and the eluate was purified using GenElute PCR DNA purification kit. The purified fragment was ligated to a pGEM-T Easy vector from Promega Corporation (Madison, Wis.). Seven clones containing the fragment were sent to the University of Michigan for DNA sequencing.

Sequence analysis demonstrated that the NZM mouse strain only expresses the B2 form CD72 (without exon 3)(SEQ. ID. NO:21). Other point mutations are most likely the result of PCR errors. This results in the protein sequence of SEQ. ID. NO: 22.

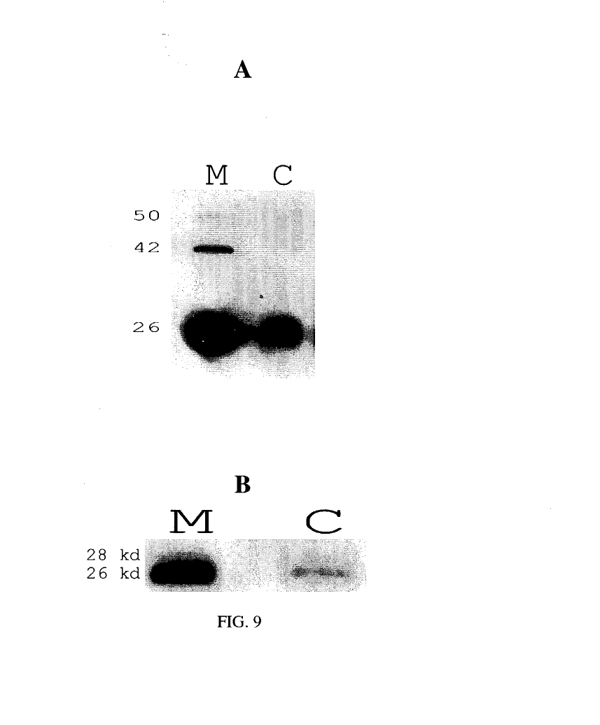

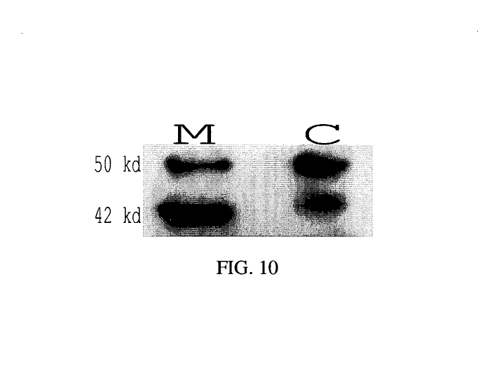

Protein lysates from mouse spleen were analyzed by Western blotting. (FIGS. 9 and 10). Mice express at least 3 types of CD72 protein products as detected by antibody against the cytoplasmic domain of the protein. The majority of the protein detected by Western Blotting are bands of 26 kd and 28 kd (FIG. 9A). The density of the 26 kd and 28 kd proteins correlates well with the amount of CD72 expressed on the cell surface (the long forms). The 28 kd protein is a truncated form of the CD72 protein with a full length cytoplasmic domain while the 26 kd protein is a truncated CD72 with a 24 amino acid deletion in the cytoplasmic domain. NZM mice express a higher amount of CD72; normal strain C57BL/6J (CD72 allele) mice express the normal lower amount of CD72. The majority of full length CD72 expressed in C57BL/6J is 50 kd protein, 42 kd CD 72 protein being the minor. In contrast, NZM mice express mainly the 42 kd protein, the 50 kd form being the minor (FIG. 10). The results are consistent with the PCR analysis.

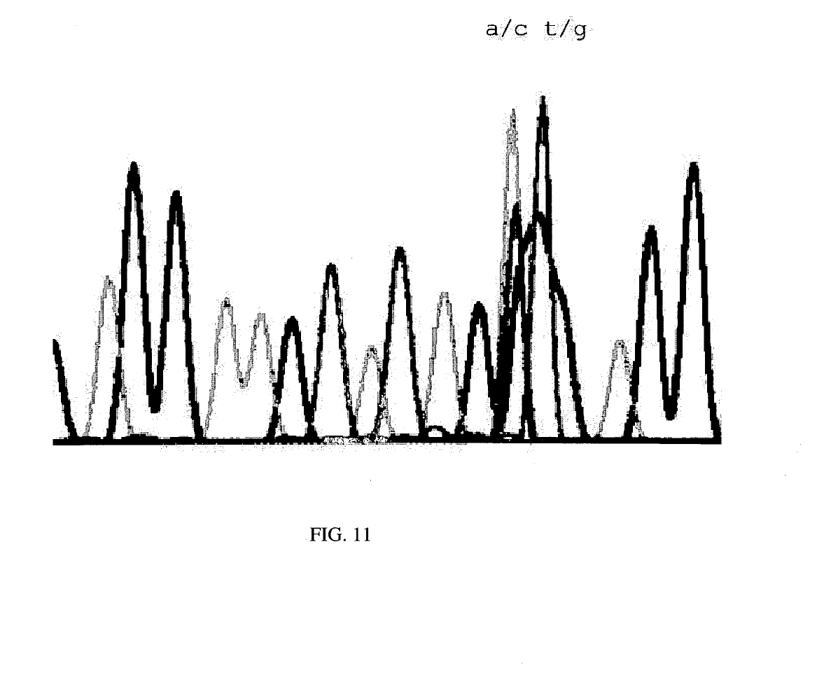

Twenty-three samples of genomic DNA from 23 normal individuals (without lupus symptoms) were analyzed by PCR analysis. The ITIM primers (SEQ. ID. NO.: 19 and SEQ. ID. NO.: 20) were used to amplify the 5′ un-transcribed region and Exons 1 and 2. No mutations in CD72 were observed. However, a patient exhibiting lupus symptoms in brain, kidney and joints, demonstrated a mutation in this region of CD72. The mutation is in the 5′ un-transcribed region. In normal patients, codons 993-994 are G-G (SEQ. ID. NO. :2). In the mutated CD72, codon 993 was either A/C/G and codon 994 was either T/G, depending on the allele (FIG. 11). The 23 normal samples represent 46 alleles in which there was no mutation.

Using methods such as those described herein, or other appropriate methods, it is now possible to diagnose diseases associated with the expression of a mutant form of CD72 by detecting the mutation or mutations in the CD72 gene that are associated with said disease.

The genomic DNA used for the diagnosis may be obtained from body cells, such as those present in peripheral blood, urine, saliva, bucca, surgical specimen, and autopsy specimens. The DNA may be used directly or may be amplified enzymatically in vitro through use of PCR (Saiki et al. Science 239:487-491 (1988)) or other in vitro amplification methods such as the ligase chain reaction (LCR) (Wu and Wallace Genomics 4:560-569 (1989)), strand displacement amplification (SDA) (Walker et al. PNAS USA 89:392-396 (1992)), self-sustained sequence replication (3SR) (Fahy et al. PCR Methods Appl. 1:25-33 (1992)), prior to mutation analysis. The methodology for preparing nucleic acids in a form that is suitable for mutation detection is well known in the art.

The detection of mutations in specific DNA sequences, such as the CD72 gene, can be accomplished by a variety of methods including, but not limited to, restriction-fragment-length-polymorphism detection based on allele-specific restriction-endonuclease cleavage (Kan and Dozy Lancet II:910-912 (1978)), hybridization with allele-specific oligonucleotide probes (Wallace et al. Nucl Acids Res 6:3543-3557 (1978)), including immobilized oligonucleotides (Saiki et al. PNAS USA 86:6230-6234 (1989)) or oligonucleotide arrays (Maskos and Southern Nucl Acids Res 21:2269-2270 (1993)), allele-specific PCR (Newton et al. Nucl Acids Res 17:2503-25 16 (1989)), mismatch-repair detection (MRD) (Faham and Cox Genome Res 5:474-482 (1995)), binding of MutS protein (Wagner et al. Nucl Acids Res 23:3944-3948 (1995)), denaturing-gradient gel electrophoresis (DGGE) (Fisher and Lerman et al. PNAS USA 80:1579-1583 (1983)), single-strand-conformation-polymorphism detection (Orita et al. Genomics 5:874-879 (1983)), RNAase cleavage at mismatched base-pairs (Myers et al. Science 230:1242 (1985)), chemical (Cotton et al. PNAS USA 85:4397-4401 (1988)) or enzymatic (Youil et al. PNAS USA 92:87-91 (1995)) cleavage of heteroduplex DNA, methods based on allele specific primer extension (Syvanen et al. Genomics 8:684-692 (1990)), genetic bit analysis (GBA) (Nikiforov et al. Nuci Acids Res 22:4167-4175 (1994)), the oligonucleotide-ligation assay (OLA) (Landegren et al. Science 241:1077 (1988)), the allele-specific ligation chain reaction (LCR) (Barrany PNAS USA 88:189-193 (1991)), gap-LCR (Abravaya et al. Nucl Acids Res 23:675-682 (1995)), and radioactive and/or fluorescent DNA sequencing using standard procedures well known in the art.

As will be appreciated, the mutation analysis may also be performed on samples of RNA by reverse transcription into cDNA therefrom. Furthermore, mutations may also be detected at the protein level using antibodies specific for the mutant and normal CD72 protein, respectively. It may also be possible to base an CD72 mutation assay on altered cellular or subcellular localization of the mutant form of the CD72 protein.

In a first method of diagnosing diseases associated with a CD72 mutation, hybridization methods such as Southern analysis, are used (see Current Protocols In Molecular Biology, Ausubel, F. et al., Eds., John Wiley & Sons, including all supplements). For example, a test sample of genomic DNA, RNA or cDNA, is obtained from an individual suspected of having (or carrying a defect for) a CD72 associated disease (the “test individual”). The individual can be an adult, child, or fetus. The test sample can be from any source which contains genomic DNA, such as a blood or tissue sample, such from skin or other organs. In a preferred embodiment, the test sample of DNA is obtained from a fibroblast skin sample, from hair roots, or from cells obtained from the oral cavity, e.g., via mouthwash. In another preferred embodiment, the test sample of DNA is obtained from fetal cells or tissue by appropriate methods, such as amniocentesis or chorionic villus sampling. The DNA, RNA, or cDNA sample is examined to determine whether one of the mutations described above is present; the presence of the mutation is indicated by hybridization of the CD72 gene in the genomic DNA, RNA, or cDNA to a nucleic acid probe. A “nucleic acid probe,” as used herein, can be a DNA probe or an RNA probe. The nucleic acid probe hybridizes to at least one of the mutations described above. A fragment of such a nucleic acid probe can also be used, provided the fragment hybridizes to the part of the CD72 gene that contains the mutation.

To diagnose CD72 associated diseases by hybridization, a hybridization sample is formed by contacting the test sample containing the CD72 gene with a nucleic acid probe. The hybridization sample is maintained under conditions which are sufficient to allow hybridization of the nucleic acid probe to the CD72 gene. Hybridization can be preformed under high stringency conditions or moderate stringency conditions, for example. “Stringency conditions” for hybridization is a term of art which refers to the conditions of temperature and buffer concentration which permit hybridization of a particular nucleic acid to another nucleic acid in which the first nucleic acid may be perfectly complimentary to the second, or the first and second nucleic acids may share only some degree of complementarity. For example, certain high stringency conditions can be used which distinguish perfectly complementary nucleic acids from those of less complementarity. “High stringency conditions” and “moderate stringency conditions” for nucleic acid hybridizations are explained in Current Protocols In Molecular Biology, supra, the teachings of which are hereby incorporated by reference. The exact conditions which determine the stringency of hybridization depend on factors such length of nucleic acids, base composition, percent and distribution of mismatch between the hybridizing sequences, temperature, ionic strength, concentration of the stabilizing agents, and other factors. Thus, high or moderate stringency conditions can be determined empirically. In one embodiment, the hybridization conditions for hybridization are moderate stringency. In a particularly preferred embodiment, the hybridization conditions for hybridization are high stringency.

Hybridization, if present, is then detected using standard methods. A hybridization occurs between the nucleic acid probe and the CD72 gene in the test sample, and the CD72 gene has a mutation corresponding to the nucleic acid probe utilized. More then one nucleic acid probe can also be concurrently used in this method. Hybridization of any one of nucleic acid probes is indicative of a mutation that is associated with the mutant CD72 associated diseases, and is therefore diagnostic for one of these diseases. For example, in the diagnosis of thyroid follicular carcinoma, a nucleic acid probe can be prepared that hybridizes to a part of the mutation of the CD72 gene giving rise to the deletion in the first ITIM structure. If this nucleic acid probe hybridizes with the CD72 gene in the test sample, a diagnosis of thyroid follicular carcinoma is made. Alternatively, a nucleic acid probe can be prepared that hybridizes to a CD72 gene having one of the other mutations described above. Hybridization of such a nucleic acid probe with the CD72 gene in the test sample is indicative of the disease associated with that particular mutation.

In another hybridization method, Northern analysis (see Current Protocols In Molecular Biology, supra) is used to identify the presence of a mutation associated with a mutant CD72 associated disease. For Northern analysis, a sample of RNA is obtained from the test individual by appropriate means. Hybridization of a nucleic acid probe, as described above, to RNA from the individual is indicative of a mutation that is associated with the mutant CD72 associated disease, and is therefore diagnostic for that disease.

The newly developed technique of nucleic acid analysis via microchip technology is also applicable to the present invention. In this technique, literally thousands of distinct oligonucleotide probes are built up in an array on a silicon chip. Nucleic acid to be analyzed is fluorescently labeled and hybridized to the probes on the chip. It is also possible to study nucleic acid-protein interactions using these nucleic acid microchips. Using this technique one can determine the presence of mutations or even sequence the nucleic acid being analyzed or one can measure expression levels of a gene of interest. The method is one of parallel processing of many, even thousands, of probes at once and can tremendously increase the rate of analysis. Several papers have been published which use this technique. Some of these are Hacia et al., Nature Genetics 14, December 1996; Shoemaker et al., 1996; Chee et al., 1996; Lockhart et al., 1996; DeRisi et al., 1996; Lipshutz et al., 1995. This method has already been used to screen people for mutations in the breast cancer gene BRCA1 (see Hacia et al., supra). This new technology has been reviewed in a news article in Chemical and Engineering News (Borman, 1996) and been the subject of an editorial (Nature Genetics, 1996).

In another method of the invention, mutation analysis by restriction digestion can be used to detect a mutation, if the mutation in the gene results in the creation or elimination of a restriction site. A test sample containing genomic DNA is obtained from the test individual. Polymerase chain reaction (PCR) or ligase chain reaction (LCR) can be used to amplify the CD72 sequence (and, if necessary, the flanking sequences) in a test sample of DNA from the test individual. RFLP analysis is conducted as described: see Current Protocols In Molecular Biology, supra. The digestion pattern of the relevant DNA fragment indicates the presence or absence of the mutation associated with the mutant CD72 associated disease.

In another method of the invention, PCR is utilized to detect the mutant gene or a lack thereof. Primers having a sequence complimentary to the sequence on either side of the mutated sequence are used to amplify the DNA or RNA (if RNA is being detected, a reverse transcriptase stage must be performed, as would be apparent to one skilled in the art) containing the mutation. Wherein the mutation results in a measurable size difference in the amplified product, the presence of the mutation can be detected by gel electrophoresis. The advantages of using the PCR reaction is that the actual mutated sequence is obtained, less starting material is required and the PCR methods allow quantitative as well as qualitative determinations to be made. Quantitative determinations allow the number of copies of a mutated gene present in a particular sample to be estimated, and given this information the severity of the disease state can be estimated.

Another alternative method for detecting the presence of the mutant gene is one in which one primer has a complimentary sequence encompassing the mutation. Amplification will therefore only occur if the mutated sequence is present. Newton et al., Nucl. Acids. Res. 17:2503 (1989). The method has previously been used in detecting mutations in the gene responsible for cystic fibrosis, and one skilled in the art could easily perform this test for the detection of the mutant gene of the present invention.

Sequence analysis can also be used to detect specific mutations in the CD72 gene. A test sample of DNA is obtained from the test individual. PCR or LCR can be used to amplify the gene, and/or its flanking sequences. The sequence of the mutant CD72 gene, or a fragment of the gene, is determined, using standard methods. The sequence of the gene (or gene fragment) is compared with the known nucleic acid sequence of the gene. The presence of any of the mutations associated with the mutant CD72 associated disease indicates that the individual is infected with, or is a carrier for that particular disease.

Analysis of the protein product of the mutant gene can also be used to detect specific mutations. With the biological molecule to be analyzed as a protein, it may be desirable to release the nucleic acid from the biological sample cells prior to protein elusion, or to remove nucleic acid from the sample eluate prior to protein analysis, thus, the sample or eluate may first be treated to release or remove the nucleic acid by mechanical disruption (such as freeze/thaw, abrasion, sonication), physical/chemical disruption such as treatment with detergents, osmotic shock, heat, enzymatic digection or nucleus treatment, all according to well known methods in the art.

Where a biological sample includes a mutant protein, the presence or absence of which is indicative of a genetic disease, the protein may be detected using conventional detection methods, for example, using protein-specific probes such as an antibody probe. Additionally, absence of the native normal protein is also indicative of a mutant gene. As such, where a genetic disease correlates with the presence or absence of an amino acid or sequence of amino acids, these amino acids may be detected using conventional means, e.g., an antibody which is specific for the native or mutant sequence.

Any of the antibody reagents useful in the method of the present invention may comprise whole antibodies, antibody fragments, polyfunctional antibody aggregates, or in general any substance comprising one or more specific binding sites from an antibody. The anti fragments may be fragments such as Fv, Fab and F(ab′).sub.2 fragments or any derivatives thereof, such as a single chain Fv fragment. The antibodies or antibody fragments may be non-recombinant, recombinant or humanized. The antibody may be of any immunoglobulin isotope, e.g., IgG, IgM and so forth. In addition, aggregates, polymers, derivatives and conjugates of immunoglobulins or their fragments can be used where appropriate. The immunoglobulin source for an antibody reagent can be obtained in any manner such as by preparation of a conventional polyclonal antiserum or by preparation of a monoclonal or a chimeric antibody. Antiserum can be obtained by well-established techniques involving immunization of an animal, such as a mouse, rabbit, guinea pig or goat, with an appropriate immunogen.

For the production of polyclonal antibodies, the peptide or polypeptide may be conjugated to a conventional carrier in order to increase its immunogenicity, and antisera to the peptide-carrier conjugate is raised in rabbits. Coupling of a peptide to a carrier protein and immunizations are performed as described (Dymecki, S. M. et al., J. Biol. Chem., 267:4815-4823 (1992)). Rabbit antibodies against this peptide are raised and the sera titered against peptide antigen by ELISA or alternatively by dot or spot blotting (Boersma and Van Leeuwen, J. Neuroscience Methods, 51:317 (1994)). At the same time, the antiserum may be used in tissue sections. The sera is shown to react strongly with the appropriate peptides by ELISA, following the procedures of Green et al., Cell, 28, 477-487 (1982). Preferably, the sera exhibiting the highest titer is subsequently used.

Techniques for preparing monoclonal antibodies are well known, and monoclonal antibodies of this invention may be prepared using a synthetic peptide, preferably bound to a carrier, as described by Arnheiter et al., Nature, 294, 278-280 (1981).

Monoclonal antibodies are typically obtained by hybridoma tissue cultures or from ascites fluid obtained from animals into which the hybridoma tissue was introduced. Nevertheless, monoclonal antibodies may be described as being “raised to” or “induced by” the synthetic peptides or their conjugates.

Particularly preferred immunological tests rely on the use of either monoclonal or polyclonal antibodies and include enzyme linked immunoassays (ELISA), immunoblotting, immunoprecipitation and radio immunoassays (RIA). (See Voller, A., Diagnostic Horizons, 2:1-7, 1978, Microbiological Associates Quarterly Publication, Walkersville, Md.; Voller, A. et al., J. Clin. Pathol., 31:507-520 (1978); U.S. Reissue Pat. No. 31,006; Butler, J. E., Meth. of Enzymol., 73:482-523 (1981); Maggio, E., Enzyme Immunoassay, CRC Press, Boca Raton, Fla. (1980); Weintraub, B., Principals of Radioimmunoassays, 7^{th} Training Course on Radio Ligandassay Techniques, The Endocrine Society, March 1986, pp. 1-5, 46-49 and 68-78). For analyzing tissues for the presence of the mutant protein of the present invention, immunohistochemistry techniques are preferably used. It will be apparent to one skilled in the art that the antibody molecule will have to be labeled to facilitate easy detection of mutant protein. Techniques for labeling antibody molecules are well known to those skilled in the art. (See Harlour and Lane, Antibodies, Cold Spring Harbor Laboratory, pp. 1-726 (1989).)

Alternatively, sandwich hybridization techniques may be used, for example, an antibody specific for a given protein. In addition, an antibody specific for a haptenic group conjugated to the binding protein can be used. Another sandwich detection system useful for detection is the avidin or streptavidin system, where a protein specific for the detectable protein has been modified by addition of biotin. In yet another embodiment, the antibody may be replaced with a non-immunoglobulin protein which has the property of binding to an immunoglobulin protein, for example, Staphylococcal protein A or Streptolococcal protein G, which are well known in the art. The protein may either itself be labeled or may be detected indirectly by a detectable labeled secondary binding protein, for example, a second antibody specific for the first antibody. Thus, if a rabbit-anti-hybrid wild-type/nonsense protein antibody serves as the first binding protein, a labeled goat-anti-rabbit immunoglobulin antibody would be a second binding protein.

In another embodiment, the signal generated by the presence of a hybrid wild-type/nonsense protein is amplified by reaction with a specific antibody for that fusion protein (e.g., an anti-beta galactosidase antibody) which is detectably labeled. One of ordinary skill in the art can devise without undue experimentation a number of such possible first and second binding protein systems using conventional methods well known in the art. Alternatively, other techniques can be used to detect the mutant proteins, including chromatographic methods such as SDS PAGE, isoelectric focusing, Western blotting, HPLC and capillary electrophoresis.

According to the present invention, a method is also provided of supplying wild-type CD72 function to a cell which carries mutant CD72 alleles. Supplying such function should suppress the hyperproliferation or hyperactivation of the recipient cells. The wild-type CD72 gene may be introduced into the cell in a vector such that the gene remains extrachromosomal. In such a situation, the gene will be expressed by the cell from the extrachromosomal location. If a gene portion is introduced and expressed in a cell carrying a mutant CD72 allele, the gene portion should encode a part of the CD72 protein which is required for non-neoplastic growth of the cell. More preferred is the situation where the wild-type CD72 gene or a part of it is introduced into the mutant cell in such a way that is recombines with the endogenous mutant CD72 gene present in the cell. Such recombination requires a double recombination event which results in the correction of the CD72 gene mutation. Vectors for introduction of genes both for recombination and for extra chromosomal maintenance are known in the art and any suitable vector may be used. Methods for introducing DNA into cells such as electroporation, calcium phosphate co-precipitation, and viral transduction are known in the art and the choice of method is within the competence of the practitioner. Cells transformed with the wild-type CD72 gene can be used as model systems to study cancer remission and drug treatments which promote such remission.

Polypeptides which have CD72 activity can be supplied to cells which carry mutant or missing CD72 alleles. The sequence of the CD72 protein is disclosed in SEQ. ID. NO:3. Protein can be produced by expression of the cDNA sequence, or a fragment of the sequence in bacteria, for example, using known expression vectors. Alternatively, CD72 can be extracted from CD72-producing mammalian cells such as pre-B cells. In addition, the techniques of synthetic chemistry can be employed to synthesize CD72 protein. Any of such techniques can provide the preparation of the present invention which comprises the CD72 gene product having the sequence shown in SEQ. ID. NO:3. The preparation is substantially free of other human proteins. This is most readily accomplished by synthesis in a microorganism or in vitro. Active CD72 molecules can be introduced into cells by microinjection or by use of liposomes, for example.

Alternatively, some such active molecules may be internalized by cells, actively or by diffusion. (See review by Ford et al. Gene Therapy 2001, Jan. 8(1): 1-4.) This process relies on the inherent property of a small number of proteins and peptides of being able to penetrate the cell membrane. The transducing property of these molecules can be conferred upon proteins which are expressed as fusions with them and thus offers an alternative to gene therapy for the delivery of therapeutic proteins into target cells.

Supply of molecules with CD72 activity should lead to a partial reversal of the hyper proliferation or hyper active state. Other molecules with CD72 activity may also be used to affect such a reversal, for example, peptides, drugs or organic compounds.

### EXAMPLE 1

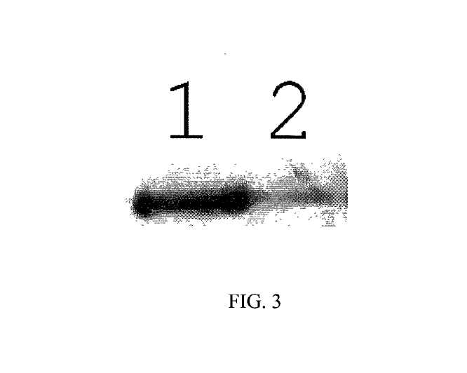

Antibody against CD72 was used to detect CD72 protein expression in tumor tissues. The loss of CD72 protein expression suggests mutations in the CD72 gene. The over-expression of CD72 also suggests mutations in the CD72 gene as the increased CD72 expression does not result in an inhibition of cell proliferation. Tissue samples from tumor tissue and the surrounding normal tissue may be procured through surgery or needle biopsy. Tissue homogenates are prepared in lysis buffer containing NP-40. The protein concentration in the homogenate can be quantified by BCA kits from Pierce Co.(Rockford, Ill.). Equal amounts of the protein from homogenates can be loaded into SDS-PAGE gels as described in FIG. 8. Alternatively, known amounts of protein from homogenates can be loaded into SDS-PAGE as described in FIG. 3. Western blotting of these electrophoresis gels is done as described in FIG. 4. The over-expression of CD72 (See FIG. 3) indicates a mutation in the CD72 from the endometrial carcinoma sample as the over-expression of CD72 does not inhibit cell growth. The loss of CD72 protein expression in lymphomas and the endometrial carcinoma of FIG. 8 also suggests a mutation in CD72 gene. Thus, we have used antibodies directed to CD72 to diagnose diseases with abnormal expression of CD72, including the cancers and lupus discussed above.

### EXAMPLE 2

A plasmid expressing the normal CD72 cDNA with a CMV promoter was introduced into an endometrial adenocarcinoma cell line ECC-1, which contains a mutation in the CD72 gene, as described above. A control vector (pCDNA3.1+ from Invitrogen, Carlsbad, Calif.) without the CD72 cDNA was used as a control. The ECC-1 cells were grown to 80% confluency. The cells were then trypsinized and washed once in complete RPMI medium. EPBS (in 250 ml of water, 2.197 g NaCl, 96.6 mg NaH2PO4.H2O, 482.4 mg Na2HPO4.7H2O) was used to suspend the cells to a concentration of approximately 5 million per ml. The cells were mixed up with DNA in the 2 mm-gap electroporation cuvette and incubated at 4 C for 15 min. Electroporation was carried out using BTX 600 electroporator (Set voltage 185V, Capacitance: 960 mF). The cuvette was put back into ice again for 15 min and then the cells were transferred to flask containing the complete RPMI medium. The medium was changed every three days. After 9 days, the cells were trypsinized and equal number of cells from both the control and test samples were put into a culture chamber. The cells were counted using XTT kits purchased from Sigma Co. (St. Louis, Mo.) The growth of the ECC-1 cell line transfected with the CD72 expressing vector was significantly slowed when compared to the vector control. This suggests that introduction of normal CD72 into cells expressing a mutant CD72 inhibits cell growth in the cancer cell line. This suggests that CD72 is a tumor suppressor gene and that wild-type CD72 can be supplied to cells that have a mutant gene resulting in a defective CD72 protein.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method of diagnosing cancer in an individual, comprising detecting a mutation in the human CD72 gene, mRNA or protein, wherein the presence of the mutation is indicative of cancer.

2. The method of claim 1, wherein the presence of the mutation in the CD72 gene is detected by direct mutation analysis by restriction digestion.

3. The method of claim 1, wherein the presence of the mutation in the CD72 gene or mRNA is detected by sequence analysis of the CD72 gene or mRNA.

4. The method of claim 1, wherein the presence of the mutation in the CD72 gene or mRNA is detected by hybridization of a nucleic acid probe to the CD72 gene or mRNA in the test sample from the individual.

5. The method of claim 1, wherein the mutation in the CD72 gene or mRNA is detected by formation of a nucleic acid duplex wherein a first strand of said duplex comprises a nucleic acid probe having a sequence complimentary to part of the gene or mRNA encompassing the mutation, and the second strand of said duplex comprises a nucleic acid sequence of the gene which is complimentary to said probe.

6. The method of claim 1, wherein the mutation is detected using PCR to amplify a fragment of the CD72 gene or mRNA encompassing the mutation, wherein the presence or absence of PCR product is indicative of the mutation.

7. The method of claim 1, wherein the mutation is detected using PCR to amplify a fragment of the CD72 gene or mRNA encompassing the mutation, wherein a PCR product that is larger or smaller than a PCR product predicted for the wild-type CD72 gene or mRNA is indicative of the mutation.

8. The method of claim 1, wherein the mutation is detected using PCR to amplify a fragment of the CD72 gene or mRNA encompassing the mutation, wherein the presence or absence of a restriction site in the PCR product is indicative of the mutation.

9. The method of claim 1, wherein the mutation is detected using PCR to amplify a fragment of the CD72 gene or mRNA encompassing the mutation, and then probing for the amplified fragment using a nucleic acid probe having a sequence complimentary to part of the CD72 gene or mRNA encompassing the mutation, or by sequencing the amplified fragment.

10. The method of claim 1, wherein the mutation is detected by detecting a difference in the relative electrophoretic mobility of a CD72 protein from an individual possessing the mutant CD72 protein, as compared to a normal CD72 protein, which is indicative of said mutation in said CD72 gene or mRNA.

11. The method of claim 1, wherein the mutant CD72 gene is detected by using antibodies which distinguish between mutant CD72 protein and normal CD72 protein.

12. The method of claim 11, wherein the antibodies are directed to mutant CD72 protein.

13. The method of claim 11, wherein the antibodies are directed to normal CD72 protein.

14. The method of claim 11, wherein the antibodies are monoclonal antibodies.

15. The method of claim 1, wherein the mutant CD72 gene protein is detected by immunocytochemistry wherein said method utilizes an antibody or antibodies which distinguish between the mutant CD72 gene protein and normal CD72 gene protein.

16. The method of claim 15, wherein the antibodies are directed to mutant CD72 protein.

17. The method of claim 15, wherein the antibodies are directed to normal CD72 protein.

18. The method of claim 1, wherein the cancer is thyroid follicular cancer.

19. The method of claim 18, wherein the mutation results in a deletion of the first ITIM structure of CD72, wherein the presence of the mutation is indicative of thyroid follicular carcinoma.

20. The method of claim 18, wherein the mutation results in a CD72 mRNA sequence with a corresponding cDNA sequence of SEQ. ID. NO:5.

21. The method of claim 18, wherein the mutation results in a mutated genomic CD72 sequence of SEQ. ID. NO:4.

22. The method of claim 18, wherein the mutation results in a CD72 protein with the sequence of SEQ. ID. NO:6.

23. The method of claim 18, wherein the mutation is detected by comparing the amount of binding of antibody specific for the mutant CD72 protein of SEQ. ID. NO:6, wherein the antibody does not bind to normal CD72.

24. A method of claim 1, wherein the cancer is renal cancer.

25. The method of claim 24, wherein the mutation results in an mRNA sequence with a corresponding cDNA sequence of SEQ. ID. NO:7, wherein the presence of the mutation is indicative of renal cancer.

26. The method of claim 24, wherein the mutation is detected by comparing the amount of binding of antibody specific for the mutant CD72 protein of SEQ. ID. NO:8, wherein the antibody does not bind to normal CD72.

27. The method of claim 1, wherein the cancer is acute lymphocytic leukemia.

28. The method of claim 27, wherein the mutation results in the deletion of codon 141 to codon 190 of SEQ. ID. NO:2, wherein the presence of the mutation is indicative of acute lymphocytic leukemia.

29. The method of claim 27, wherein the mutation results in an mRNA sequence with a corresponding cDNA sequence which contains the sequence of SEQ. ID. NO:11.

30. The method of claim 27, wherein the mutation results in an mRNA sequence with a corresponding cDNA sequence which contains the sequence of SEQ. ID. NO:12.

31. The method of claim 27, wherein the mutation results in an mRNA sequence with a corresponding cDNA sequence which contains the sequence of SEQ. ID. NO:13.

32. The method of claim 27, wherein the mutation results in an mRNA sequence with a corresponding cDNA sequence which contains the sequence of SEQ. ID. NO:14.

33. The method of claim 27, wherein the mutation results in a protein with the sequence of SEQ. ID. NO:16.

34. The method of claim 27, wherein the mutation is detected by comparing the amount of binding of antibody specific for the mutant CD72 protein of SEQ. ID. NO:16, wherein the antibody does not bind to normal CD72.

35. The method of claim 1, wherein the cancer is endometrial adenocarcinoma.

36. The method of claim 36, wherein the mutation results in a CD72 mRNA with a corresponding cDNA sequence of SEQ ID NO:10.

37. The method of claim 35, wherein the mutation results in a mutated genomic CD72 sequence of SEQ ID NO:9.

38. The method of claim 1, wherein the cancer is chondrosarcoma.

39. The method of claim 38, wherein the mutation results in a CD72 mRNA with a corresponding cDNA sequence of SEQ ID NO:17.

40. The method of claim 38, wherein the mutation results in a genomic CD72 sequence of SEQ ID NO:18.

41. The method of claim 1, wherein the cancer is ovarian cancer.

42. The method of claim 1, wherein the cancer is lymphoma.

43. A method of diagnosing lupus erythematosus in an individual, comprising detecting a mutation in the human CD72 gene, mRNA or protein, wherein the presence of the mutation is indicative of lupus erythematosus.

44. The method of claim 43, wherein the presence of the mutation in the CD72 gene is detected by direct mutation analysis by restriction digestion.

45. The method of claim 43, wherein the presence of the mutation in the CD72 gene is detected by sequence analysis of the CD72 gene or mRNA.

46. The method of claim 43, wherein the presence of the mutation in the CD72 gene or mRNA is detected by hybridization of a nucleic acid probe to the CD72 gene or mRNA in the test sample from the individual.

47. The method of claim 43, wherein the CD72 mutation is detected by formation of a nucleic acid duplex wherein a first strand of said duplex comprises a nucleic acid probe having a sequence complimentary to part of the gene or mRNA encompassing the mutation, and the second strand of said duplex comprises a nucleic acid sequence of the gene or mRNA which is complimentary to said probe.

48. The method of claim 43, wherein the mutation is detected using PCR to amplify a fragment of the CD72 gene or mRNA encompassing the mutation, wherein the presence or absence of PCR product is indicative of the mutation.

49. The method of claim 43, wherein the mutation is detected using PCR to amplify a fragment of the CD72 gene or mRNA encompassing the mutation, wherein a PCR product that is larger or smaller than a PCR product predicted for the wild-type CD72 gene or mRNA is indicative of the mutation.

50. The method of claim 43, wherein the mutation is detected using PCR to amplify a fragment of the CD72 gene or mRNA encompassing the mutation, wherein the presence or absence of a restriction site in the PCR product is indicative of the mutation.

51. The method of claim 43, wherein the mutation is detected using PCR to amplify a fragment of the CD72 gene or mRNA encompassing the mutation, and then probing for the amplified fragment using a nucleic acid probe having a sequence complimentary to part of the gene or mRNA encompassing the mutation, or by sequencing the amplified fragment.

52. The method of claim 43, wherein the mutation is detected by detecting a difference in the relative electrophoretic mobility of a CD72 protein from an individual possessing a mutant CD72 protein as compared to a normal CD72 protein which is indicative of said mutation in said CD72 gene or mRNA.

53. The method of claim 43, wherein the mutant CD72 gene protein is detected by immunoblotting using antibodies which distinguish between mutant CD72 gene protein and normal CD72 gene protein.

54. The method of claim 43, wherein the mutant CD72 gene protein is detected by a monoclonal antibody, wherein said monoclonal antibody distinguishes between mutant CD72 protein and normal CD72 protein.

55. The method of claim 43, wherein the mutant CD72 gene protein is detected by immunocytochemistry wherein said method utilizes an antibody or antibodies which distinguish between the mutant CD72 gene protein and normal CD72 gene protein.

56. The method of claim 43, wherein the mutation is detected by comparing the amount of binding of antibody specific for the mutant CD72 protein of SEQ. ID. NO: 22, wherein the antibody does not bind to normal CD72.

57. The method of claim 43, wherein the mutation results in an mRNA with a corresponding CD72 cDNA sequence of SEQ ID NO:21.

58. An antibody specific for a mutant CD72 protein, wherein the antibody does not bind to normal CD72 protein.

59. The antibody of claim 58, which antibody is a monoclonal antibody.

60. The antibody of claim 58, wherein the mutant CD72 protein has the sequence of SEQ. ID. NO:6.

61. The antibody of claim 58, wherein the mutant CD72 protein has the sequence of SEQ. ID. NO:8.

62. The antibody of claim 58, wherein the mutant CD72 protein has the sequence of SEQ. ID. NO:16.

63. The antibody of claim 58, wherein the mutant CD72 protein has the sequence of SEQ. ID. NO:22.

64. A method of supplying wild-type CD72 gene function to a cell which has lost said gene function by virtue of a mutation in a CD72 gene, comprising:
introducing a wild-type CD72 gene into a cell which has lost said gene function such that said wild-type CD72 gene is expressed in the cell.

65. The method of claim 64, wherein the wild-type CD72 gene introduced recombines with the endogenous mutant CD72 gene present in the cell by a double recombination event to correct the CD72 gene mutation.

66. The method of claim 64, wherein the mutation is a deletion of the first ITIM domain of said CD72 gene.

67. The method of claim 64, wherein the mutation results in a CD72 mRNA with a sequence corresponding to the cDNA sequence of SEQ ID NO:5.

68. The method of claim 64, wherein the mutation is a deletion of codon 141 to codon 190 of SEQ ID. NO:2.

69. The method of claim 64, wherein the mutation results in a CD72 mRNA with a sequence corresponding to the cDNA sequence of SEQ ID NO:10.

70. The method of claim 64, wherein the mutation results in a CD72 mRNA with a sequence corresponding to the cDNA sequence of SEQ ID NO:7.

71. A method of supplying wild-type CD72 gene function to a cell which has a mutation in a CD72 gene, comprising:
introducing a portion of the wild-type CD72 gene into a cell which has lost said gene function such that said portion is expressed in the cell, said portion comprising a part of the wild-type CD72 gene which contains the mutation in the cell.

72. A method of supplying wild-type CD72 protein function to a cell which has lost said protein function by virtue of a mutation in a CD72 gene, comprising:
introducing wild-type CD72 protein into a cell which has lost said protein function.
